(19) United States
(12) Patent Application Publication
(10) Pub. No.: US 2025/0163029 A1
(43) Pub. Date: May 22, 2025

(54) PYRIDONE OR PYRIMIDONE DERIVATIVE AND APPLICATION THEREOF

(71) Applicant: Medshine Discovery Inc., Nanjing, Jiangsu (CN)

(72) Inventors: Shuhui Chen, Shanghai (CN); Jiajie Feng, Shanghai (CN); Zheng Wang, Shanghai (CN); Peng Li, Shanghai (CN); Haiying He, Shanghai (CN)

(73) Assignee: Medshine Discovery Inc., Nanjing, Jiangsu (CN)

(21) Appl. No.: 18/839,378

(22) PCT Filed: Feb. 16, 2023
(86) PCT No.: PCT/CN2023/076449

§ 371 Date: Oct. 21, 2024

(30) Foreign Application Priority Data
Feb. 18, 2022 (CN) 202210155270.4
Mar. 18, 2022 (CN) 202210273298.8
Mar. 21, 2022 (CN) 202210286278.4
Apr. 19, 2022 (CN) 202210417721.7
May 19, 2022 (CN) 202210559569.6

(51) Int. Cl. C07D 403/14; A61K 31/513; A61P 31/14; C07D 401/14; C07D 403/12

(52) U.S. Cl. CPC C07D 403/14; A61K 31/513; A61P 31/14; C07D 401/14; C07D 403/12

(57) ABSTRACT

Disclosed in the present invention are a pyridone or pyrimidone derivative and an application thereof, and particularly disclosed are a compound as shown in formula (VI) and a pharmaceutically acceptable salt thereof.

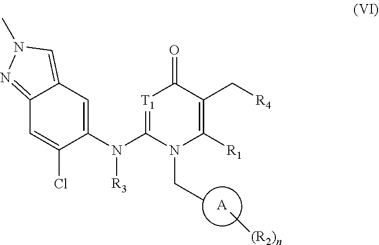

### CROSS REFERENCE TO RELATED APPLICATIONS

This application is a National Stage filing under 35 U.S.C. 371 of International PCT Application No. PCT/CN2023/076449, filed on Feb. 16, 2023, which claims:

The priority and benefits of Chinese Patent Application No. 202210155270.4 filed with the China National Intellectual Property Administration on Feb. 18, 2022, the priority and benefits of Chinese Patent Application No. 202210273298.8 filed with the China National Intellectual Property Administration on Mar. 18, 2022, the priority and benefits of Chinese Patent Application No. 202210286278.4 filed with the China National Intellectual Property Administration on Mar. 21, 2022, the priority and benefits of Chinese Patent Application No. 202210417721.7 filed with the China National Intellectual Property Administration on Apr. 19, 2022, and the priority and benefits of Chinese Patent Application No. 202210559569.6 filed with the China National Intellectual Property Administration on May 19, 2022. The contents disclosed in each of these applications are incorporated herein by reference in their entirety.

### TECHNICAL FIELD

The present disclosure relates to the technical field of pharmaceutical chemistry, in particular to pyridone or pyrimidone derivatives and applications thereof, and specifically to a compound represented by formula (VI) and a pharmaceutically acceptable salt thereof.

### BACKGROUND

The COVID-19 pandemic caused by the novel coronavirus (SARS-CoV-2) has caused millions of deaths and threatened public health and safety. Although COVID-19 vaccines have been used worldwide, the virus continues to mutate. There is still a urgent need for effective oral antiviral drugs.

SARS-CoV-2 main protease (3CL protease), as a key enzyme for coronavirus replication, is considered to be an important target for the treatment of diseases caused by various coronaviruses, including COVID-19. SARS-CoV-2 main protease (3CL protease) can hydrolyze viral polyproteins to produce other functional proteins. Many 3CL protease inhibitors have been developed and applied in preclinical and clinical studies now. The development of 3CL protease inhibitors is of great value and significance for the prevention and treatment of coronavirus infections.

### SUMMARY

The present disclosure provides a compound represented by formula (VI) or a pharmaceutically acceptable salt thereof, which is selected from:

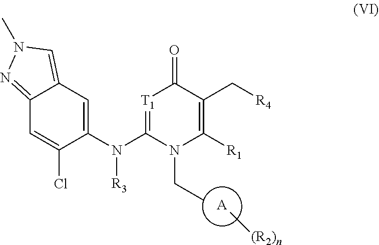

wherein
R₁ is selected from H, F, Cl, Br, I and C₁₋₃ alkyl, wherein the C₁₋₃ alkyl is optionally substituted with 1, 2 or 3 halogens;
each R₂ is independently selected from H, F, Cl, Br, I and C₁₋₃ alkyl, wherein the C₁₋₃ alkyl is optionally substituted with 1, 2 or 3 halogens;
Ring A is selected from phenyl and 5- to 6-membered heteroaryl;
R₃ is selected from H and C₁₋₄ alkyl;
R₄ is selected from

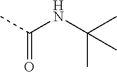

and 5-membered heteroaryl, wherein the 5-membered heteroaryl is optionally substituted with 1 or 2 Rₐ;
each Rₐ is independently selected from F, Cl, Br, I, C₁₋₃ alkyl and C₃₋₆ cycloalkyl, wherein the C₁₋₃ alkyl and C₃₋₆ cycloalkyl are optionally substituted with 1, 2, or 3 halogens;
n is selected from 1, 2, 3, 4 and 5;
T₁ is selected from CH and N.

In some embodiments of the present disclosure, each of the above Rₐ is independently selected from H, F, —CH₃, —CF₃, cyclopropyl and cyclobutyl, and other variables are as defined in the present disclosure.

In some embodiments of the present disclosure, each of the above Rₐ is independently selected from H, F, —CH₃, —CF₃ and cyclobutyl, and other variables are as defined in the present disclosure.

In some embodiments of the present disclosure, each of the above Rₐ is independently selected from H and —CH₃, and other variables are as defined in the present disclosure.

In some embodiments of the present disclosure, the above R₁ is selected from H, F, —CH₃ and —CF₃, and other variables are as defined in the present disclosure.

In some embodiments of the present disclosure, each of the above R₂ is independently selected from H, F, —CH₃ and —CF₃, and other variables are as defined in the present disclosure.

In some embodiments of the present disclosure, the above R₃ is selected from H and —CH₃, and other variables are as defined in the present disclosure.

In some embodiments of the present disclosure, the above R₄ is selected from

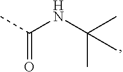

pyrrolyl, imidazolyl, pyrazolyl and triazolyl, wherein the pyrrolyl, imidazolyl, pyrazolyl and triazolyl are optionally substituted with 1 or 2 Rₐ, and other variables are as defined in the present disclosure.

In some embodiments of the present disclosure, the above R₄ is selected from

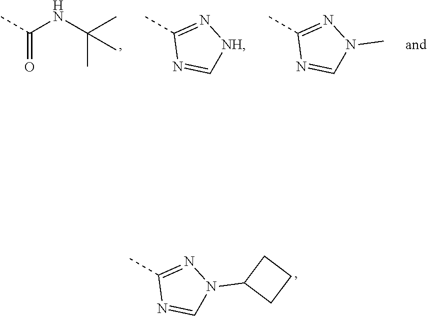

and other variables are as defined in the present disclosure.

In some embodiments of the present disclosure, the above R₄ is selected from

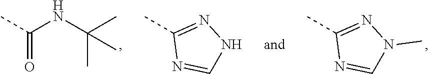

and other variables are as defined in the present disclosure.

In some embodiments of the present disclosure, the above R₄ is selected from

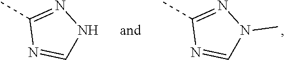

and other variables are as defined in the present disclosure.

In some embodiments of the present disclosure, the above ring A is selected from phenyl, pyridyl, pyrimidinyl, pyridazinyl, pyrazolyl, pyrrolyl and imidazolyl, and other variables are as defined in the present disclosure.

In some embodiments of the present disclosure, the above ring A is selected from phenyl,

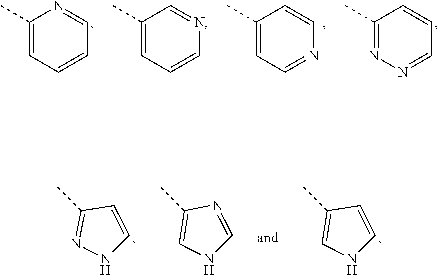

and other variables are as defined in the present disclosure.

In some embodiments of the present disclosure, the above-mentioned structural moiety

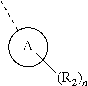

is selected from

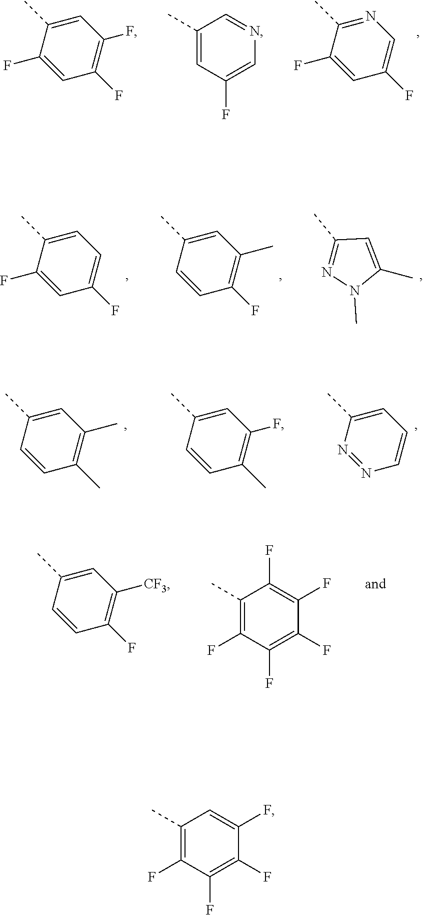

and other variables are as defined in the present disclosure.

In some embodiments of the present disclosure, provided herein is the above-mentioned compound or a pharmaceutically acceptable salt thereof, wherein the compound has the structure shown in formula (VI-1):

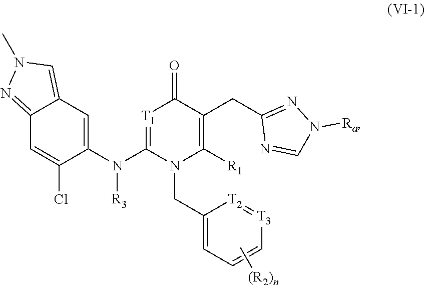

wherein T₂ and T₃ are each independently selected from CH and N; and other variables are as defined in the present disclosure.

The present disclosure also provides a compound represented by formula (I) or a pharmaceutically acceptable salt thereof, which is:

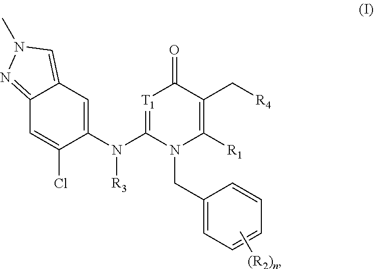

wherein
R₁ is selected from H, F, Cl, Br and C₁₋₃ alkyl, wherein the C₁₋₃ alkyl is optionally substituted with 1, 2 or 3 halogens;
each R₂ is independently selected from H, F, Cl and Br;
R₃ is selected from H and C₁₋₄ alkyl;
R₄ is 5-membered heteroaryl, wherein the 5-membered heteroaryl is optionally substituted with 1 or 2 Rₐ;
each Rₐ is independently selected from F, Cl, Br and C₁₋₃ alkyl;
n is selected from 1, 2 and 3;
T₁ is selected from CH and N.

In some embodiments of the present disclosure, the above R₁ is selected from H, —CH₃ and —CF₃, and other variables are as defined in the present disclosure.

In some embodiments of the present disclosure, each of the above R₂ is independently selected from H and F, and other variables are as defined in the present disclosure.

In some embodiments of the present disclosure, the above R₃ is selected from H and —CH₃, and other variables are as defined in the present disclosure.

In some embodiments of the present disclosure, the above R₄ is selected from pyrrolyl, imidazolyl, pyrazolyl and triazolyl, wherein the pyrrolyl, imidazolyl, pyrazolyl and triazolyl are optionally substituted with 1 or 2 Rₐ, and other variables are as defined in the present disclosure.

In some embodiments of the present disclosure, the above R₄ is

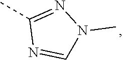

and other variables are as defined in the present disclosure.

Some other embodiments of the present disclosure are obtained by arbitrarily combining the above variables.

The present disclosure also provides the following compounds or pharmaceutically acceptable salts thereof, which are selected from the structures represented by formula (I-1) and (I-2):

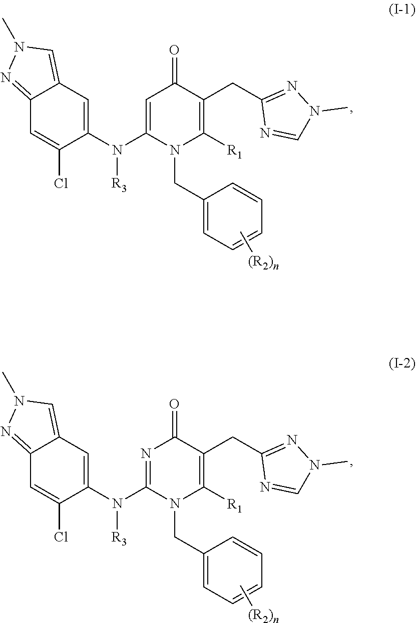

wherein R₁, R₂, R₃, R₄ and n are as defined in the present disclosure.

The present disclosure also provides the following compounds or pharmaceutically acceptable salts thereof, which are selected from:

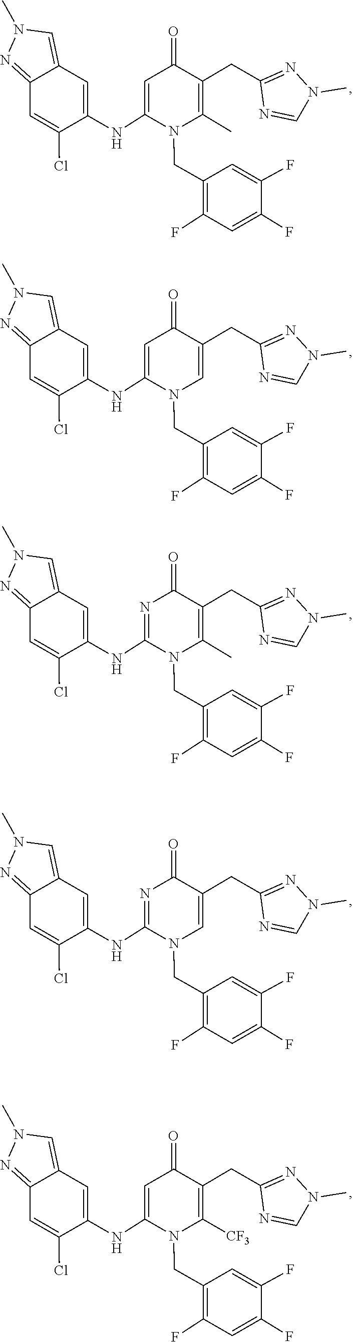

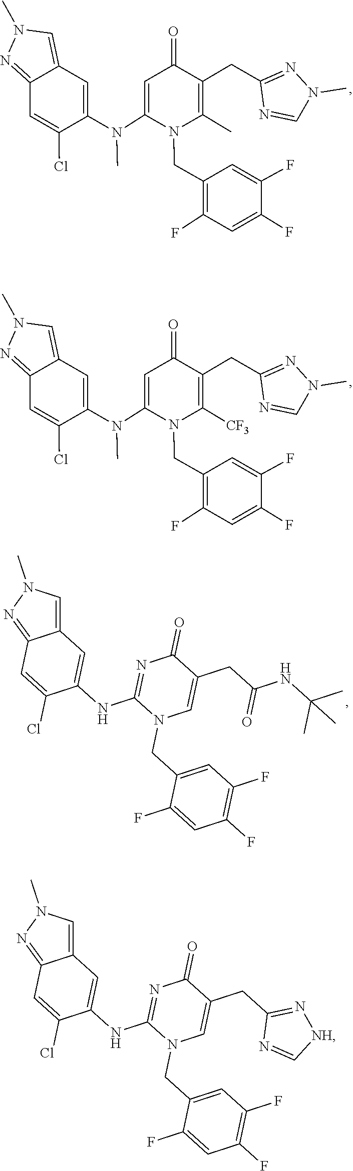

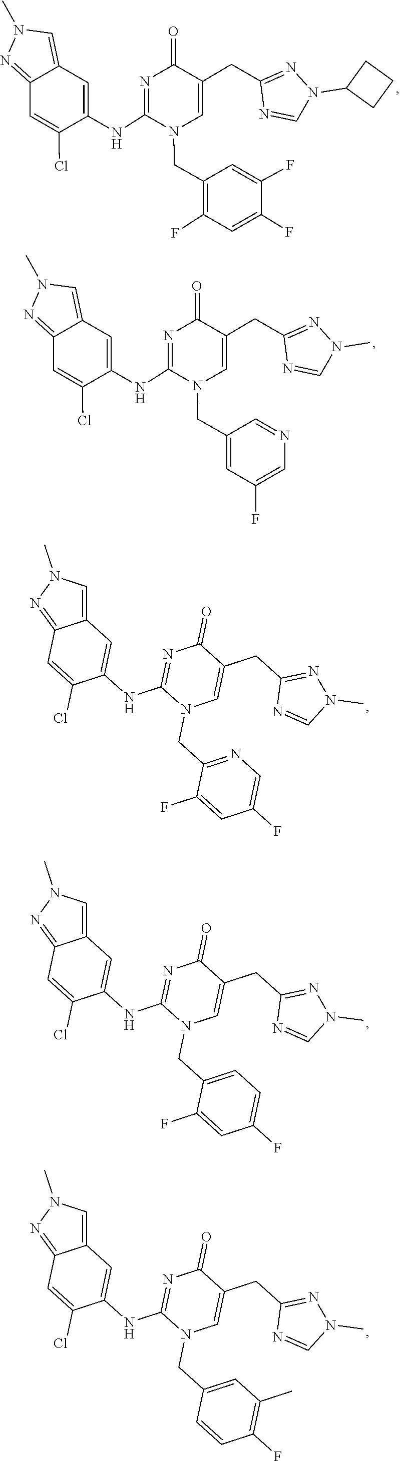

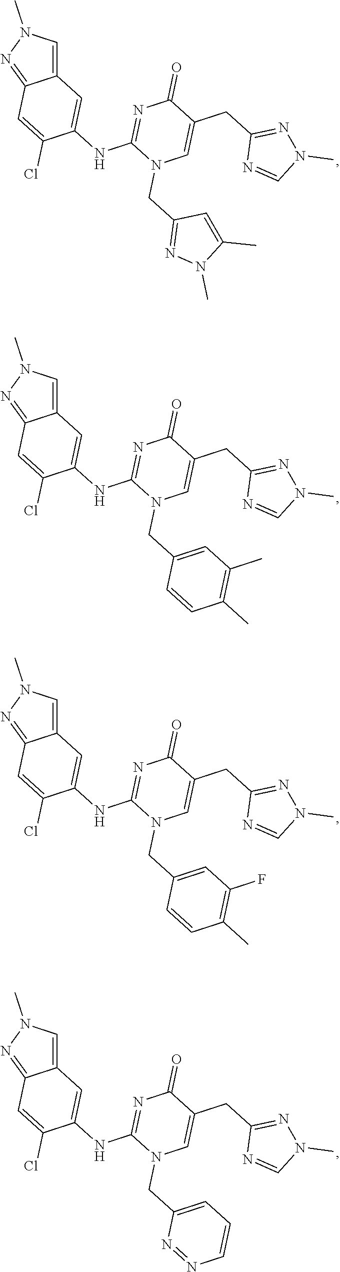 [SMILES: Cc1cc(Cn2cc(Cc3ncn(C)n3)c(=O)nc2Nc2cc3cn(C)nc3cc2Cl)nn1C ; Cc1ccc(Cn2cc(Cc3ncn(C)n3)c(=O)nc2Nc2cc3cn(C)nc3cc2Cl)cc1C ; Cc1ccc(Cn2cc(Cc3ncn(C)n3)c(=O)nc2Nc2cc3cn(C)nc3cc2Cl)cc1F ; Cn1cnc(Cc2cn(Cc3cccnn3)c(Nc3cc4cn(C)nc4cc3Cl)nc2=O)n1]

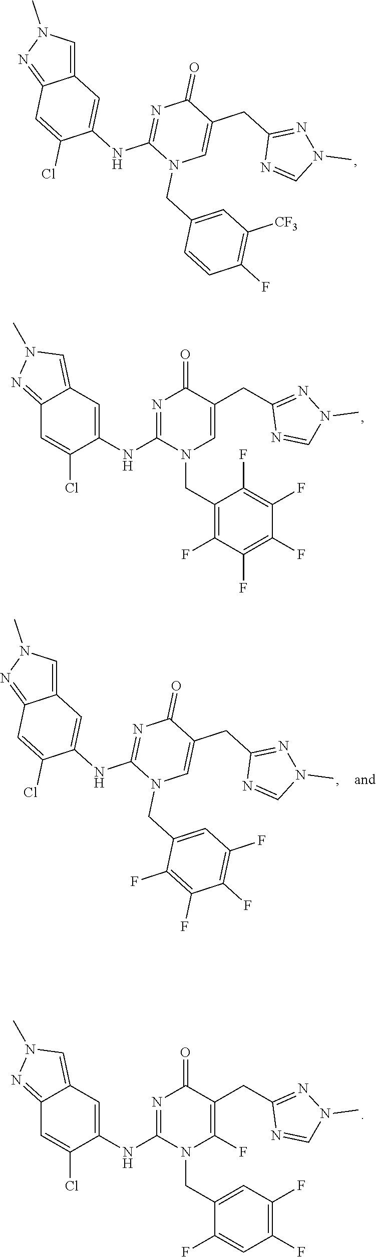

In some embodiments of the present disclosure, the use of the above-mentioned compound or a pharmaceutically acceptable salt thereof in the manufacture of a medicament for treating novel coronavirus infection is also provided.

The present disclosure also provides the following synthetic route:

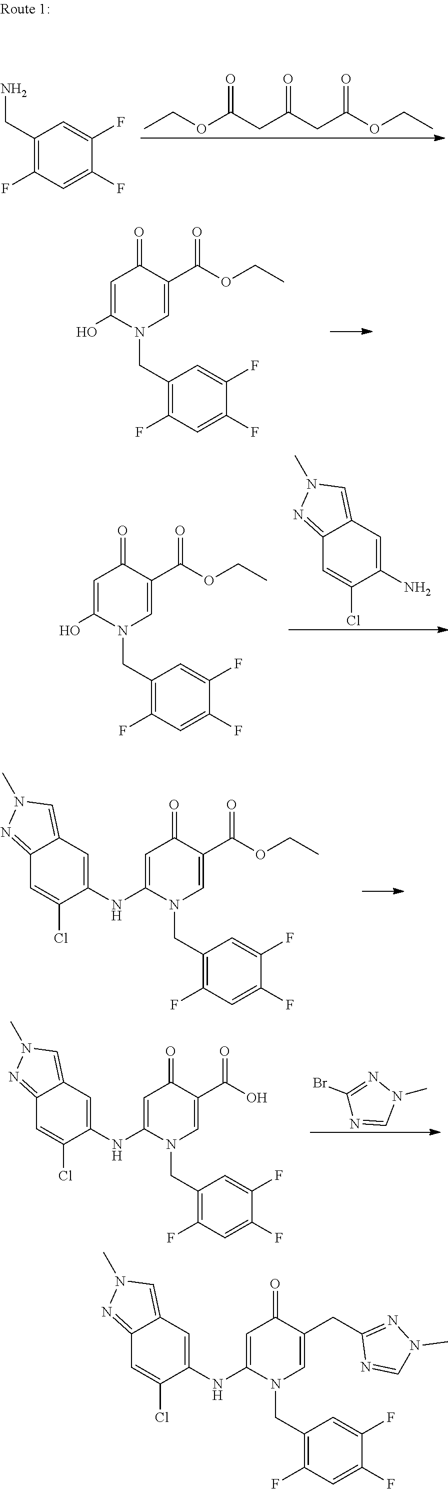

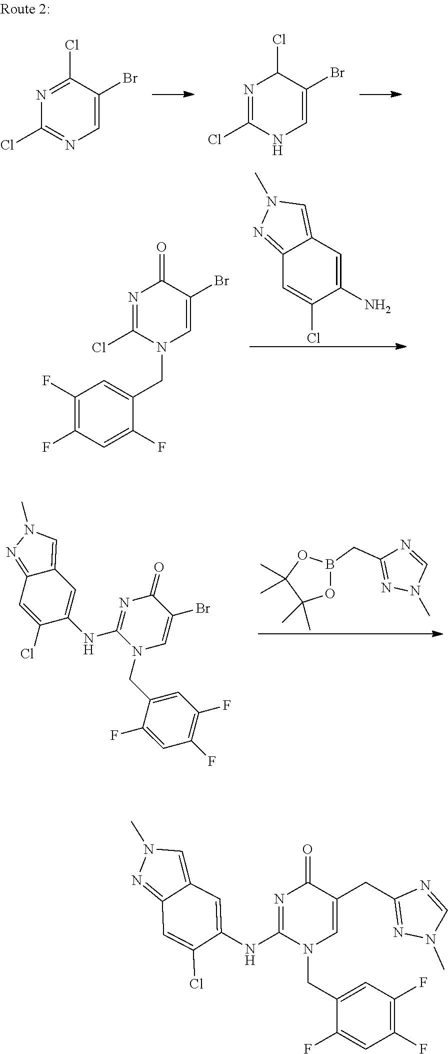

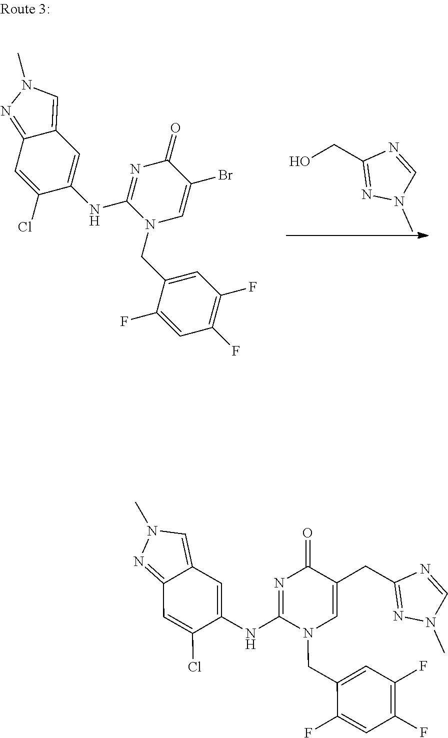

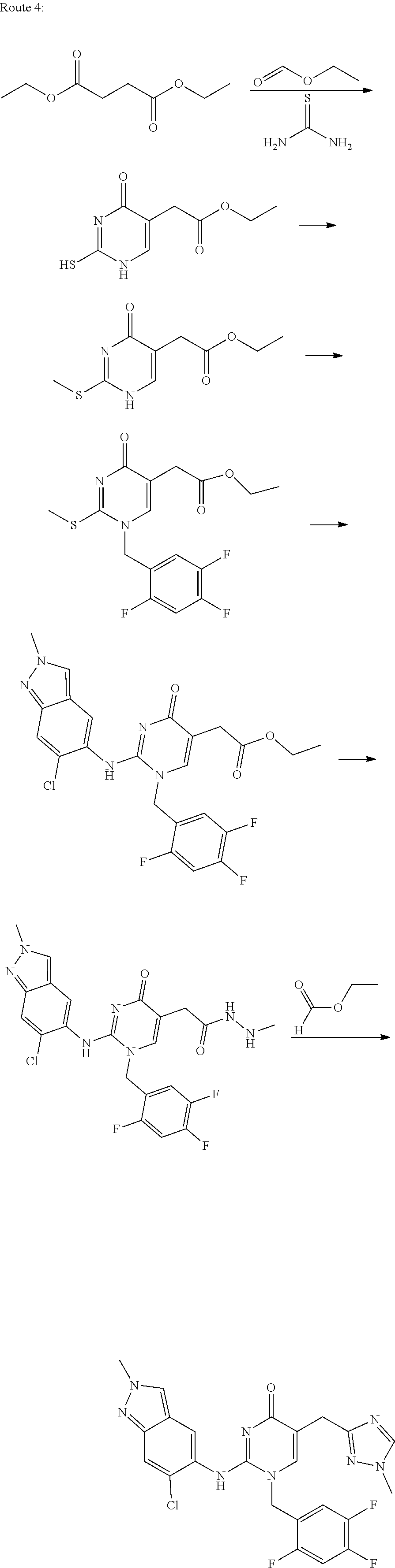

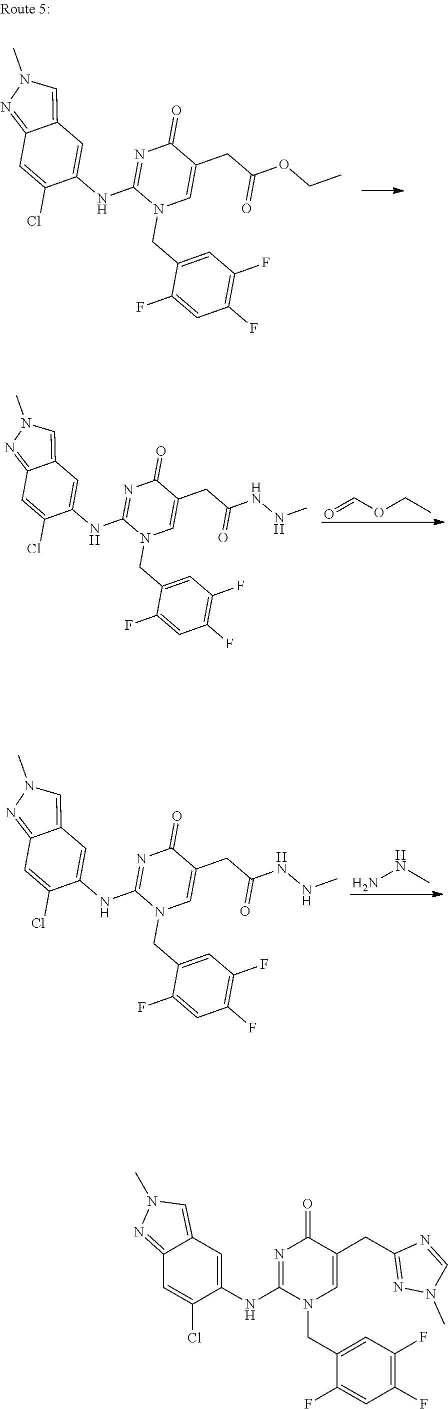

The present disclosure also provides the following assay method:

### Assay Method 1: Evaluation of the In Vitro Anti-Novel Coronavirus Mpro Protease Activity of the Assay Compounds

1. Assay materials:

### 1.1 Reagents and Consumables:

TABLE 1
Names and brands of reagents and consumables

| | Reagents and consumables | Brand |
|---|---|---|
| 1 | Tris | Sigma |
| 2 | EDTA | Sigma |
| 3 | NaCl | Sigma |
| 4 | 384 well Plate | Perkin Elmer |
| 5 | Dimethyl sulfoxide (DMSO) | Sigma |
| 6 | Substrate (Dabcyl- | GenScript |
| | KTSAVLQSGFRKM-(Edans)) | |
| 7 | SARS-CoV-2 Mpro | WuXi AppTec |
| 8 | GC376 | TargetMol |

### 1.2 Instrument:

TABLE 2
Instruments and brands

| | Instrument | Brand |
|---|---|---|
| 1 | SpectraMax M2e microplate | Molecular Devices |
| | reader | |
| 2 | Echo 655 liquid workstation | Labcyte |
| 3 | Tabletop high-speed centrifuge | Eppendorf |

2. Assay methods:

The compound was dissolved in DMSO, and diluted in a 3-fold gradient with Echo655 according to the concentration requirements for the assay to 10 concentration points. Duplicate assays were set at each concentration. The diluted solution was added to a 384-well plate. Mpro protein and substrate were diluted with assay buffer (100 mM NaCl, 20 mM Tris-HCl, 1 mM EDTA). Mpro protein was added to the 384-well assay plate, and incubated with the compound for 30 min at room temperature. Then the substrate was added. The assay concentration of Mpro protein was 25 nM, and the assay concentration of substrate was 25 μM. After incubating for 60 minutes in a 30° C. constant temperature incubator, the fluorescence signal value of Ex/Em=340 nm/490 nm was detected by microplate reader. At the same time, the background well containing the substrate and compound but not containing Mpro protein was detected as control.

### 3. Data Analysis:

1) The inhibition rate was calculated using the following formula:

$$Inhibition rate \% = [ ( Compound - {BG}_{Compound} ) - ( ZPE - {BG}_{ZPE} ) ] /$$

$$[ ( HPE - {BG}_{HPE} ) - ( ZPE - {BG}_{ZPE )} ] * 100 \%$$

^{#}HPE: 100% inhibition control, containing 25 nM Mpro protein+25 μM substrate+1 μM GC376
ZPE: No-inhibition control, containing 25 nM Mpro protein+25 μM substrate, not containing compound
Compound: Assay compound well, containing 25 nM Mpro protein+25 μM substrate+compound
BG: Background control well, containing 25 μM substrate+compound, not containing Mpro protein

2) Log (agonist) vs. response—variable slope nonlinear fitting analysis was carried out on the inhibition rate data (inhibition rate %) of the compound by using GraphPad Prism software, and the IC₅₀ value of the compound was obtained.

### Assay Method 2: Evaluation of In Vitro Anti-Coronavirus Activity of Compounds Using a Cytopathic Model

### 1. Assay Materials

### 1.1. Reagents and Consumables

TABLE 3
Names and brands of reagents and consumables

| | Names of Reagents and consumables | Brand |
|---|---|---|
| 1 | MEM medium | Sigma |
| 2 | L-Glutamine | Gibco |
| 3 | Non-essential amino acid | Gibco |
| 4 | Double antibody (Penicillin- | HyClone |
| | Streptomycin Solution) | |
| 5 | Fetal bovine serum (FBS) | ExCell |
| 6 | Phosphate buffered saline (DPBS) | Corning |
| 7 | 0.25% Trypsin | Gibco |
| 8 | CellTiter Glo Cell Viability Assay Kit | Promega |
| 9 | Remdesivir | MCE |
| 10 | 96-well plate | Grenier |

### 1.2. Instruments

TABLE 4
Instruments and brands

| | Instrument | Brand |
|---|---|---|
| 1 | Microplate reader | BioTek |
| 2 | Cell counter | Beckman |
| 3 | CO₂ incubator | Thermo |

### 1.3. Cells and Viruses

MRC5 cells and coronavirus HCoV OC43 were purchased from ATCC.

MRC5 cells were cultured in MEM (Sigma) medium supplemented with 10% fetal bovine serum (Excell), 1% double antibody (Hyclone), 1% L-glutamine (Gibco) and 1% non-essential amino acids (Gibco). MEM (Sigma) medium supplemented with 5% fetal bovine serum (Excell), 1% double antibody (Hyclone), 1% L-glutamine (Gibco) and 1% non-essential amino acid (Gibco) was used as the assay culture medium.

### 2. Assay Method

TABLE 5
Virus assay methods used in this study

| | | Compound | | |
|---|---|---|---|---|
| | | treatment | | |
| | | time (day)/ | | |
| | | endpoint | Control | Detection |
| Virus (strain) | Cell | method | compound | Reagent |
| HCoV OC43, | 20,000 MRC5 | 5/CPE | Remdesivir | CellTiter |
| 100TCID₅₀/well | cells/well | | | Glo. |

Cells were inoculated into a 96 microwell plate at a certain density (Table 5) and cultured overnight in an incubator at 5% CO₂ and 37° C. On the second day, the compound diluted in multiples was added (8 concentration points, duplicate wells), 50 μL per well. Then the diluted virus was added to the cells at 100 TCID₅₀ per well, 50 μL per well. Cell control (cell without compound treatment or virus infection), virus control (cell infected with virus without compound treatment) and culture medium control (only culture medium) were set. The final volume of the assay culture medium was 200 μL, and the final concentration of DMSO in the culture medium was 0.5%. Cells were cultured in a 5% CO₂, 33° C. incubator for 5 days. Cell viability was detected using the cell viability assay kit CellTiter Glo (PROMEGA). Cytotoxicity assay was performed under the same conditions as the antiviral assay, but without virus infection.

### 3. Data Analysis:

The antiviral activity and cytotoxicity of compounds were represented by the inhibition rate (%) and cell viability (%) of compounds with different concentrations on the cytopathic effect caused by the virus, respectively. The calculation formula is as follows:

$$Inhibition rate ( \% ) = ( reading value of assay well - average value of virus control ) / ( average value of cell control - average value of virus control ) \times 100$$

$$Cell viability ( \% ) =$$

$$( reading value of assay well - average value of culture medium control ) /$$

$$( average value of cell control -$$

$$average value of culture medium control ) \times 100$$

GraphPad Prism was used to perform nonlinear fitting analysis on the inhibition rate and cell viability of compounds, and to calculate the half effective concentration (EC₅₀) and half cytotoxic concentration (CC₅₀) of the compounds.

### Technical Effects

The covalent binding of the compound of the present disclosure to the main protease of SARS-CoV-2 can effectively block the further replication of the virus; the compound of the present disclosure has better in vitro anti-novel coronavirus Mpro protease activity, better in vitro anti-coronavirus and anti-SARS-CoV-2 virus activity at the cellular level, and no cytotoxicity. The pharmacokinetic properties of the compound of the present disclosure meet the requirements of drug formulation. The compound of the present disclosure has a higher free drug exposure amount and a higher bioavailability in animals, and exhibits a protective effect in an efficacy assay in an animal model.

### Definitions and Terms

Unless otherwise specified, the following terms and phrases used herein are intended to have the following meanings. A specific term or phrase should not be considered indefinite or unclear in the absence of a particular definition, but should be understood in the conventional sense. When a trade name appears herein, it is intended to refer to its corresponding commodity or active ingredient thereof.

The term “pharmaceutically acceptable” is used herein in terms of those compounds, materials, compositions, and/or dosage forms, which are suitable for use in contact with human and animal tissues within the scope of reliable medical judgment, with no excessive toxicity, irritation, allergic reaction or other problems or complications, commensurate with a reasonable benefit/risk ratio.

The term “pharmaceutically acceptable salt” refers to a salt of the compound disclosed herein that is prepared by reacting the compound having a specific substituent disclosed herein with a relatively non-toxic acid or base. When the compound disclosed herein contains a relatively acidic functional group, a base addition salt can be obtained by bringing the neutral form of the compound into contact with a sufficient amount of base in a pure solution or a suitable inert solvent. The pharmaceutically acceptable base addition salt includes a salt of sodium, potassium, calcium, ammonium, organic amine or magnesium or similar salts. When the compound disclosed herein contains a relatively basic functional group, an acid addition salt can be obtained by bringing the neutral form of the compound into contact with a sufficient amount of acid in a pure solution or a suitable inert solvent. Certain specific compounds disclosed herein contain both basic and acidic functional groups and can be converted to any base or acid addition salt.

The pharmaceutically acceptable salt disclosed herein can be prepared from the parent compound that contains an acidic or basic moiety by conventional chemical methods. Generally, such salt can be prepared by reacting the free acid or base form of the compound with a stoichiometric amount of an appropriate base or acid in water or an organic solvent or a mixture thereof.

Unless otherwise specified, the term “isomer” is intended to include geometric isomers, cis-trans isomers, stereoisomers, enantiomers, optical isomers, diastereomers and tautomers.

The compound disclosed herein may be present in a specific geometric or stereoisomeric form. The present disclosure contemplates all such compounds, including cis and trans isomer, (−)- and (+)-enantiomer, (R)- and (S)-enantiomer, diastereomer, (D)-isomer, (L)-isomer, and racemic mixture and other mixtures, for example, an enantiomer or diastereomer enriched mixture, all of which are encompassed within the scope disclosed herein. The substituent such as alkyl may have an additional asymmetric carbon atom. All these isomers and mixtures thereof are encompassed within the scope disclosed herein.

Unless otherwise specified, the term “enantiomer” or “optical isomer” refers to stereoisomers that are in a mirrored relationship with each other.

Unless otherwise specified, the term “cis-trans isomer” or “geometric isomer” is produced by the inability of a double bond or a single bond between ring-forming carbon atoms to rotate freely.

Unless otherwise specified, the term “diastereomer” refers to a stereoisomer which has two or more chiral centers in a molecule and is in a non-mirrored relationship between molecules.

Unless otherwise specified, “(+)” means dextroisomer, “(−)” means levoisomer, and “(t)” means racemate.

Unless otherwise specified, a wedged solid bond ( [symbol] ) and a wedged dashed bond ( [symbol] ) indicate the absolute configuration of a stereocenter; a straight solid bond ( [symbol] ) and a straight dashed bond ( [symbol] ) indicate the relative configuration of a stereocenter; a wavy line ( [symbol] ) indicates a wedged solid bond ( [symbol] ) or a wedged dashed bond ( [symbol] ); or a wavy line ( [symbol] ) indicates a straight solid bond ( [symbol] ) or a straight dashed bond ( [symbol] ).

[symbol]

Unless otherwise specified, the term “enriched in one isomer”, “isomer enriched”, “enriched in one enantiomer” or “enantiomeric enriched” means that the content of one isomer or enantiomer is less than 100%, and the content of the isomer or enantiomer is 60% or more, or 70% or more, or 80% or more, or 90% or more, or 95% or more, or 96% or more, or 97% or more, 98% or more, 99% or more, 99.5% or more, 99.6% or more, 99.7% or more, 99.8% or more, or 99.9% or more.

Unless otherwise specified, the term “isomer excess” or “enantiomeric excess” refers to the difference between the relative percentages of two isomers or two enantiomers. For example, if one isomer or enantiomer is present in an amount of 90% and the other isomer or enantiomer is present in an amount of 10%, the isomer or enantiomeric excess (ee value) is 80%.

Optically active (R)- and (S)-isomer, or D and L isomer can be prepared using chiral synthesis or chiral reagents or other conventional techniques. If one kind of enantiomer of certain compound disclosed herein is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form a salt of the diastereomeric isomer which is then subjected to diastereomeric resolution through the conventional method in the art to give the pure enantiomer. In addition, the enantiomer and the diastereomer are generally isolated through chromatography which uses a chiral stationary phase and optionally combines with a chemical derivative method (for example, carbamate generated from amine).

The compounds disclosed herein may contain an unnatural proportion of atomic isotopes at one or more of the atoms that make up the compounds. For example, a compound may be labeled with a radioisotope such as tritium (³H), iodine-125 (¹²⁵I) or C-14 (¹⁴C). For another example, hydrogen can be replaced by heavy hydrogen to form a deuterated drug. The bond between deuterium and carbon is stronger than that between ordinary hydrogen and carbon. Compared with undeuterated drugs, deuterated drugs have advantages of reduced toxic side effects, increased drug stability, enhanced efficacy, and prolonged biological half-life of drugs. All changes in the isotopic composition of compounds disclosed herein, regardless of radioactivity, are included within the scope of the present disclosure.

The term “optional” or “optionally” means that the subsequently described event or circumstance may occur but does not necessarily occur, and that the description includes instances where said event or circumstance occurs and instances where said event or circumstance does not occur.

The term “substituted” means that one or more than one hydrogen atom (s) on a specific atom are substituted by a substituent, including deuterium and hydrogen variants, as long as the valence of the specific atom is normal and the substituted compound is stable. When the substituent is oxo (i.e., ═O), it means two hydrogen atoms are substituted. Positions on an aromatic ring cannot be substituted by oxo. The term “optionally substituted” means an atom can be substituted by a substituent or not, unless otherwise specified, the species and number of the substituent may be arbitrary so long as being chemically achievable.

When any variable (such as R) occurs in the constitution or structure of the compound more than once, the definition of the variable at each occurrence is independent. Thus, for example, if a group is substituted by 0-2 R, the group can be optionally substituted by up to two R, wherein the definition of R at each occurrence is independent. Moreover, a combination of the substituent and/or the variant thereof is allowed only when the combination results in a stable compound.

When the number of a linking group is 0, such as —(CRR)₀—, it means that the linking group is a single bond.

When the number of a substituent is 0, it means that the substituent does not exist. For example, -A-(R)₀ means that the structure is actually -A.

When a substituent is vacant, it means that the substituent does not exist. For example, when X in A-X is vacant, it means that the structure is actually A.

When one of the variables is a single bond, it means that the two groups linked by the single bond are connected directly. For example, when L in A-L-Z represents a single bond, the structure of A-L-Z is actually A-Z.

When the bond of a substituent can be cross-linked to two or more atoms on a ring, such a substituent can be bonded to any atom on the ring, for example, the structural unit

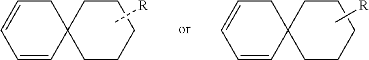

means that the substitution with substituent R can occur at any position on cyclohexyl or cyclohexadiene. When an enumerative substituent does not indicate through which atom it is linked to the substituted group, such substituent can be bonded through any of its atoms. For example, a pyridyl group as a substituent may be linked to the substituted group through any one of carbon atoms on the pyridine ring.

When an enumerative linking group does not indicate its linking direction, its linking direction is arbitrary. For example, when the linking group L in

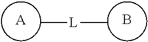

is -M-W—, the -M-W— can be linked to the ring A and the ring B in the same direction as the reading order from left to right to constitute

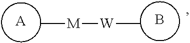

or can be linked to the ring A and the ring B in the reverse direction as the reading order from left to right to constitute

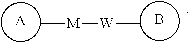

A combination of the linking groups, substituents and/or variants thereof is allowed only when such combination can result in a stable compound.

Unless otherwise specified, when a group has one or more connectable sites, any one or more sites of the group can be connected to other groups through chemical bonds. Where the connection position of the chemical bond is variable, and there is H atom(s) at a connectable site(s), when the connectable site(s) having H atom(s) is connected to the chemical bond, the number of H atom(s) at this site will correspondingly decrease as the number of the connected chemical bond increases, and the group will become a group of corresponding valence. The chemical bond between the site and other groups can be represented by a straight solid bond ( [symbol] ), a straight dashed bond ( [symbol] ), or a wavy line

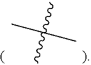

For example, the straight solid bond in —OCH₃ indicates that the group is connected to other groups through the oxygen atom in the group; the straight dashed bond in

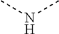

indicates that the group is connected to other groups through two ends of the nitrogen atom in the group; the wavy line in

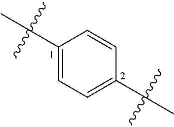 [SMILES: *c1ccc(*)cc1]

indicates that the group is connected to other groups through the 1- and 2-carbon atoms in the phenyl group.

Unless otherwise specified, the term “halo” or “halogen”, by itself or as part of another substituent, refers to a fluorine, chlorine, bromine, or iodine atom.

Unless otherwise specified, the term “C₁₋₄ alkyl” is used to indicate a linear or branched saturated hydrocarbon group consisting of 1 to 4 carbon atoms. The C₁₋₄ alkyl group includes C₁₋₂, C₁₋₃, and C₂₋₃ alkyl groups, and the like. It may be monovalent (e.g., methyl), divalent (e.g., methylene) or multivalent (e.g., methenyl). Examples of C₁₋₄ alkyl groups include, but are not limited to, methyl (Me), ethyl (Et), propyl (including n-propyl and isopropyl), butyl (including n-butyl, isobutyl, s-butyl and t-butyl), and the like.

Unless otherwise specified, the term “C₁₋₃ alkyl” is used to indicate a linear or branched saturated hydrocarbon group consisting of 1 to 3 carbon atoms. The C₁₋₃ alkyl group includes C₁₋₂ and C₂-3 alkyl groups and the like. It may be monovalent (e.g., methyl), divalent (e.g., methylene) or multivalent (e.g., methenyl). Examples of C₁₋₃ alkyl groups include, but are not limited to, methyl (Me), ethyl (Et), propyl (including n-propyl and isopropyl), and the like.

Unless otherwise specified, the number of atoms in a ring is generally defined as the membered number of the ring, for example, “5- to 7-membered ring” refers to a “ring” having 5 to 7 atoms arranged around.

Unless otherwise specified, Cₙ₋ₙ₊ₘ or Cₙ-Cₙ₊ₘ includes any one of n to n+m carbons. For example, C₁₋₁₂ includes C₁, C₂, C₃, C₄, C₅, C₆, C₇, C₈, C₉, C₁₀, C₁₁, and C₁₂. Cₙ₋ₙ₊ₘ or Cₙ-Cₙ₊ₘ also includes any range of n to n+m. For example, C₁₋₁₂ includes C₁₋₃, C₁₋₆, C₁₋₉, C₃₋₆, C₃₋₉, C₃₋₁₂, C₆₋₉, C₆₋₁₂, C₉₋₂, and the like. Similarly, the n-membered to n+m-membered ring means that the number of atoms on the ring is n to n+m. For example, 3- to 12-membered ring includes 3-membered ring, 4-membered ring, 5-membered ring, 6-membered ring, 7-membered ring, 8-membered ring, 9-membered ring, 10-membered ring, 11-membered ring, and 12-membered ring. The n-membered to n+m-membered ring also means that the number of atoms on the ring includes any range from n to n+m. For example, 3- to 12-membered ring includes 3- to 6-membered ring, 3- to 9-membered ring, 5- to 6-membered ring, 5- to 7-membered ring, 6- to 7-membered ring, 6- to 8-membered ring, and 6- to 10-membered ring, and the like.

Unless otherwise specified, “C₃₋₆ cycloalkyl” means a saturated cyclic hydrocarbon group composed of 3 to 6 carbon atoms, which is a monocyclic or bicyclic system, and the C₃₋₆ cycloalkyl includes C₃₋₅, C₄₋₅ and C₅₋₆ cycloalkyl, etc.; it can be monovalent, divalent or multivalent. Examples of C₃₋₆ cycloalkyl include, but are not limited to, cyclopropyl, cyclobutyl, cyclopentyl, cyclohexyl, and the like.

Unless otherwise specified, the terms “5- to 6-membered heteroaromatic ring” and “5- to 6-membered heteroaryl” may be used interchangeably. The term “5- to 6-membered heteroaryl” means a monocyclic group having a conjugated pi electron system and consisting of 5 to 6 ring atoms, of which 1, 2, 3 or 4 ring atoms are heteroatoms independently selected from O, S, N, P, and Se, and the remainder atoms are carbon atoms, wherein the carbon atom may be substituted with oxo (C═O), the nitrogen atom is optionally quaternized and the nitrogen and sulfur heteroatoms are optionally oxidized (i.e., NO and S(O)ₚ, p is 1 or 2). The 5- to 6-membered heteroaryl group may be attached to the remainder of a molecule by a heteroatom or a carbon atom. The 5- to 6-membered heteroaryl group includes 5-membered and 6-membered heteroaryl groups. Examples of the 5- to 6-membered heteroaryl group include, but are not limited to, pyrrolyl (including N-pyrrolyl, 2-pyrrolyl, 3-pyrrolyl, and the like), pyrazolyl (including 2-pyrazolyl and 3-pyrazolyl, and the like), imidazolyl (including N-imidazolyl, 2-imidazolyl, 4-imidazolyl, and 5-imidazolyl, and the like), oxazolyl (including 2-oxazolyl, 4-oxazolyl, and 5-oxazolyl, and the like), triazolyl (1H-1,2,3-triazolyl, 2H-1,2,3-triazolyl, 1H-1,2,4-triazolyl and 4H-1,2,4-triazolyl, and the like), tetrazolyl, isoxazolyl (3-isoxazolyl, 4-isoxazolyl and 5-isoxazolyl, and the like), thiazolyl (including 2-thiazolyl, 4-thiazolyl and 5-thiazolyl, and the like), furyl (including 2-furyl and 3-furyl, and the like), thienyl (including 2-thienyl and 3-thienyl, and the like), pyridyl (including 2-pyridyl, 3-pyridyl and 4-pyridyl, and the like), pyrazinyl or pyrimidinyl (including 2-pyrimidinyl and 4-pyrimidinyl, and the like).

Unless otherwise specified, the terms “5-membered heteroaromatic ring” and “5-membered heteroaryl” may be used interchangeably. The term “5-membered heteroaryl” means a cyclic group having a conjugated pi electron system and composed of 5 ring atoms, in which 1, 2, 3 or 4 ring atoms are heteroatoms independently selected from O, S, N, P, and Se, and the remainder is carbon atom, wherein the carbon atom may be substituted with oxo (C═O), the nitrogen atom is optionally quaternized and the nitrogen and sulfur heteroatoms may be optionally oxidized (i.e., NO and S(O)ₚ, p is 1 or 2). A 5-membered heteroaryl can be attached to the remainder of a molecule through a heteroatom or a carbon atom. Examples of 5-membered heteroaryl include, but are not limited to, pyrrolyl (including N-pyrrolyl, 2-pyrrolyl, 3-pyrrolyl, and the like), pyrazolyl (including 2-pyrazolyl and 3-pyrazolyl, and the like), imidazolyl (including N-imidazolyl, 2-imidazolyl, 4-imidazolyl, and 5-imidazolyl, and the like), oxazolyl (including 2-oxazolyl, 4-oxazolyl, and 5-oxazolyl, and the like), triazolyl (1H-1,2,3-triazolyl, 2H-1,2,3-triazolyl, 1H-1,2,4-triazolyl and 4H-1,2,4-triazolyl, and the like), tetrazolyl, isoxazolyl (3-isoxazolyl, 4-isoxazolyl and 5-isoxazolyl, and the like), thiazolyl (including 2-thiazolyl, 4-thiazolyl and 5-thiazolyl, and the like), furyl (including 2-furyl and 3-furyl, and the like), thienyl (including 2-thienyl and 3-thienyl, and the like).

The compound disclosed herein can be prepared by a variety of synthetic methods well known to the skilled in the art, including the following enumerative embodiment, the embodiment formed by the following enumerative embodiment in combination with other chemical synthesis methods and the equivalent replacement well known to the skilled in the art. The alternative embodiment includes, but is not limited to the embodiment disclosed herein.

The structures of the compounds of the present disclosure can be confirmed by conventional methods well known to those skilled in the art. If the present disclosure relates to an absolute configuration of a compound, the absolute configuration can be confirmed by conventional techniques in the art, such as single crystal X-Ray diffraction (SXRD). In the single crystal X-Ray diffraction (SXRD), the diffraction intensity data of the cultivated single crystal is collected using a Bruker D8 venture diffractometer with a light source of CuKα radiation in a scanning mode of ^{φ/ω} scan; after collecting the relevant data, the crystal structure is further analyzed by the direct method (Shelxs97) to confirm the absolute configuration.

All the solvents used in the present disclosure are commercially available.

Compounds are named according to general naming principles in the art or by ChemDraw® software, and commercially available compounds are named with their vendor directory names.

### BRIEF DESCRIPTION OF DRAWINGS

FIG. 1. Induced integration of S-217622 into 7L0D;

FIG. 2. The results of covalent docking of compound 1 with 7L0D;

FIG. 3. The results of covalent docking of compound 2 with 7L0D;

FIG. 4. The results of covalent docking of compound 3 with 7L0D;

FIG. 5. The results of covalent docking of compound 4 with 7L0D.

### DETAILED DESCRIPTION

The present disclosure is described in detail below by means of examples. However, it is not intended that these examples have any disadvantageous limitations to the present disclosure.

The present disclosure has been described in detail herein, and the embodiments are also disclosed herein. It will be apparent to those skilled in the art that various changes and modifications may be made to the embodiments disclosed herein without departing from the spirit and scope disclosed herein.

### Calculation Example 1. Prediction of the Binding Mode of the Compounds of the Present Disclosure with the Main Protease of SARS-CoV-2

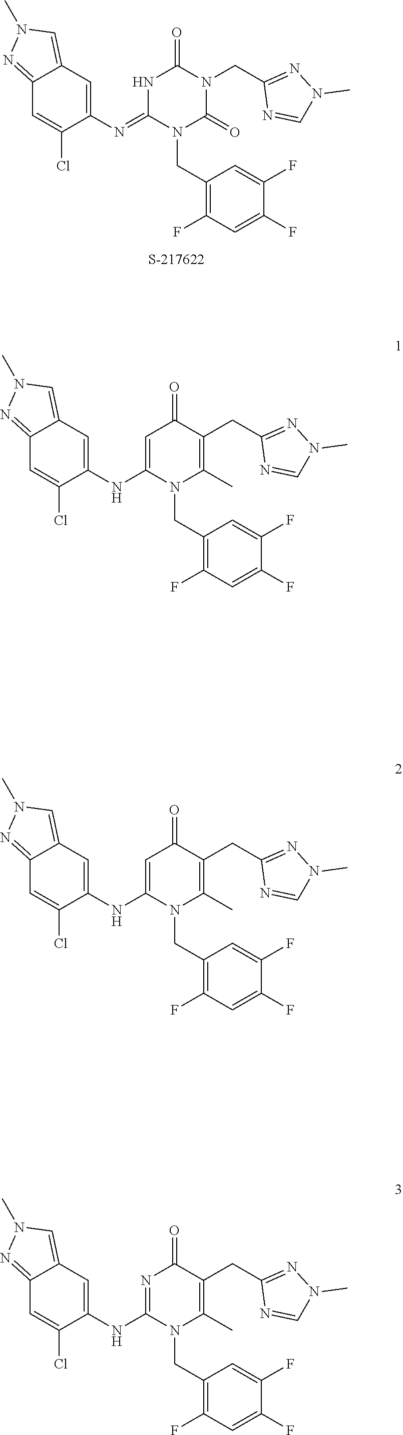

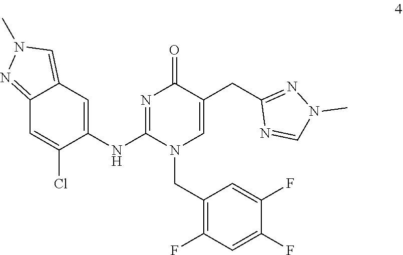

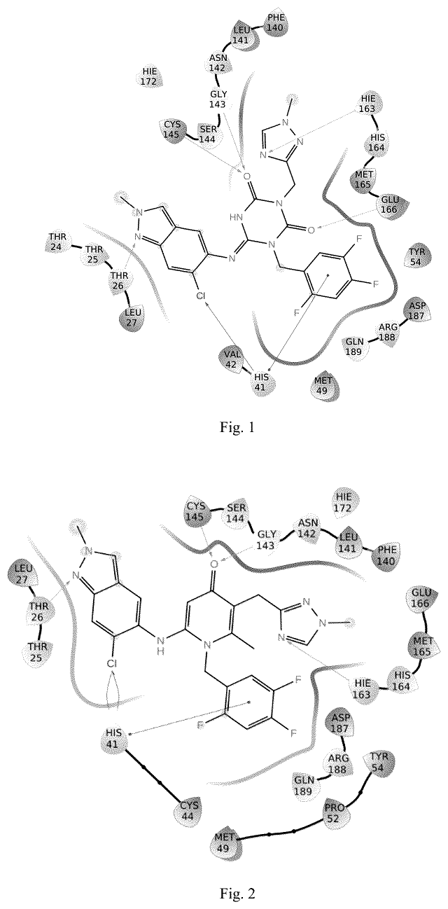

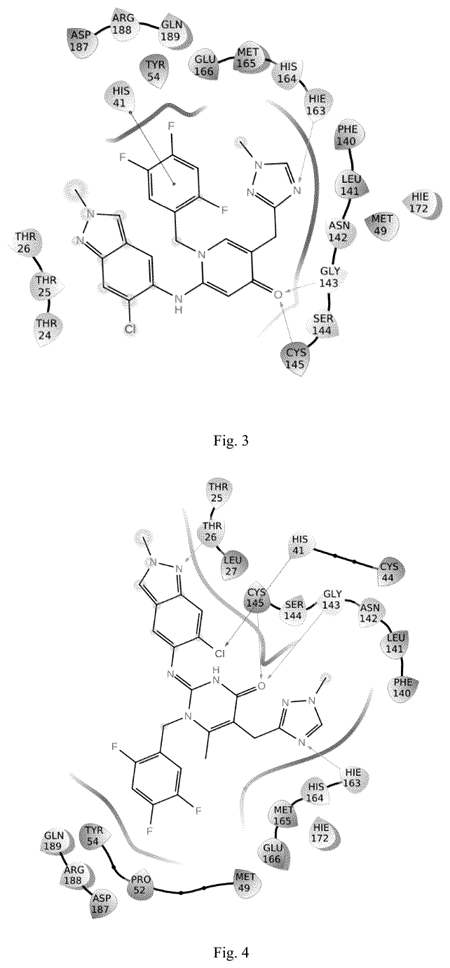

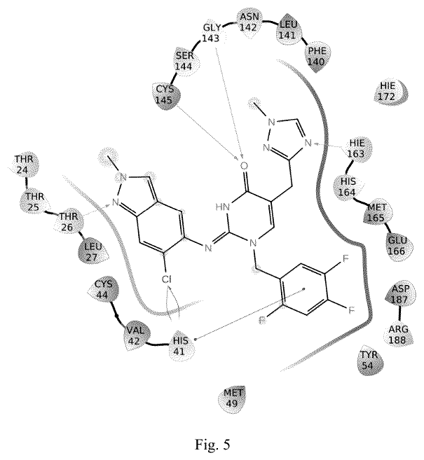

SARS-CoV-2 main protease cocrystal structure (PDB ID code: 7L0D) was used as a docking template in the binding mode prediction. To prepare the protein, the protein preparation wizard module of Maestro^{[1]} was used to add hydrogen atoms and the OPLS4 force field was used. The cocrystal structure was optimized for H-bond. Water molecules outside 3 Å of the cocrystal small molecule were removed, and the overall energy was optimized. First, the molecule S-217622 disclosed by Shionogi was docked to reproduce the reported binding mode. Since the small molecule in the cocrystal structure is somewhat different from the molecular series we docked, the Induced Fit Docking^{[2]} and Protocol: Standard options in Maestro (Schrödinger version 2021-2) were first used to dock the molecule to be docked into the prepared 7L0D structure, and the best binding model was selected, as shown in FIG. 1. The selected model maintained the main hydrogen bonding between the original cocrystal small molecule and the protein. A 30 Å docking grid was generated with the center of mass of the compound in this binding model, and a docking model was generated using the Glide^{[3]} Receptor Grid Generation module. For the preparation of ligands: the 3D structure of the newly designed molecules was generated and LigPrep was used to minimize energy^{[4]}. Based on this docking model, the newly designed molecules were docked using the SP docking mode in Glide^{[4]}. The binding modes of compounds 1 to 4 are shown in FIGS. 2 to 5.
[1] Maestro, Schrödinger, LLC, New York, NY, 2021.
[2] Induced Fit Docking protocol; Glide, Schrödinger, LLC, New York, NY, 2021; Prime, Schrödinger, LLC, New York, NY, 2021.
[3] Glide, Schrödinger, LLC, New York, NY, 2021.
[4] LigPrep, Schrödinger, LLC, New York, NY, 2021.

Conclusion: The compounds of the present disclosure have better binding to the main protease of SARS-CoV-2, and well-reproduced the binding mode of positive reference molecule S-217622: this series of molecules form hydrogen bonds with Hie163 at P1, with Thr26 at P1′, and with Glu166 and Gly143 at the core. In addition, the trifluorobenzene ring fragment forms a π-π bond with His41 at P2. The docking score of the newly designed molecule is close to or better than that of the positive reference molecule, and the non-covalent binding to the SARS-CoV-2 main protease will effectively block the further replication of the virus.

### Example 1

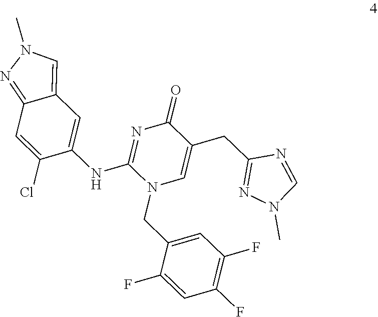 [SMILES: Cn1cnc(Cc2cn(Cc3cc(F)c(F)cc3F)c(Nc3cc4cn(C)nc4cc3Cl)nc2=O)n1]

### Synthetic Route 1:

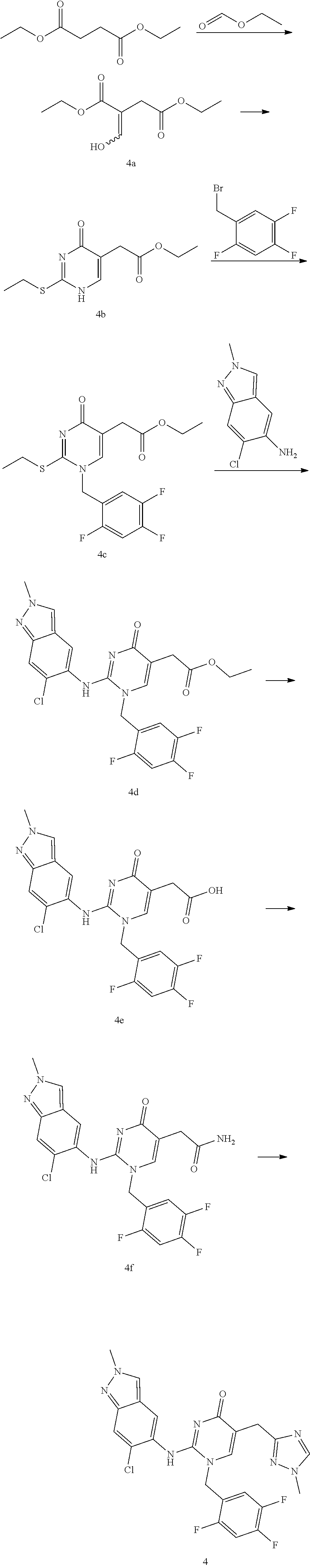

### Step 1: Synthesis of Compound 4a

Toluene (30 mL) and sodium ethoxide (7.01 g, 102.94 mmol) were added to a dry 100 mL single-neck bottle, and a mixed solution of diethyl succinate (15 g, 86.11 mmol, 14.42 mL) and ethyl formate (7.67 g, 103.47 mmol, 8.32 mL) was added dropwise under an ice bath. After the addition was completed, the mixture was stirred at 20° C. for 22 hours. After the reaction was completed, 30 mL of ice water was slowly added, and the mixture was stirred for 50 min. The layers were separated. The toluene layer was extracted twice with water (50 mL). The combined aqueous phases were adjusted to pH 2-3 with concentrated hydrochloric acid, and stirred for 20 min. The aqueous phase was extracted three times with ethyl acetate (50 mL). The combined organic phases were washed with saturated brine (50 mL). The organic phases were concentrated by rotary evaporation to dryness to obtain the crude product 4a, which was directly used in the next step.

### Step 2: Synthesis of Compound 4b

4a (16 g, 79.13 mmol) and S-ethylisothiourea hydrobromide (14.65 g, 79.13 mmol) were weighed and dissolved in water (71 mL). 18 mL of aqueous sodium hydroxide (4.75 g, 118.69 mmol) was added to the above solution and the mixture was stirred at 100° C. for 16 hours. After the reaction was cooled to room temperature, acetic acid was added dropwise to the reaction solution to adjust the pH to neutral. The mixture was stirred for another 10 min. The precipitated solid was filtered by suction, and washed with water (200 mL). The solid was dissolved in acetonitrile, and rotary evaporated to dryness to obtain 4b.

### Step 3: Synthesis of Compound 4c

4b (5.8 g, 23.94 mmol) was weighed and dissolved in dichloromethane (90 mL). Diisopropylethylamine (4.64 g, 35.91 mmol, 6.25 mL) and (2,4,5-trifluorophenyl)methylbromide (6.46 g, 28.73 mmol) were added and the mixture was stirred at 20° C. for 24 hours. After the reaction was completed, the reaction solution was concentrated by rotary evaporation to dryness, and the crude product was separated by column chromatography (petroleum ether/ethyl acetate=1:0-1:1) to obtain 4c.

### Step 4: Synthesis of Compound 4d

4c (0.5 g, 1.29 mmol) and 6-chloro-2-methyl-2H-indazol-5-amine (305.53 mg, 1.68 mmol) were weighed and dissolved in tert-butyl alcohol (5 mL), and acetic acid (357.30 mg, 1.29 mmol, 1.6 mL) was added. The mixture was stirred at 100° C. for 72 hours. After the reaction was completed, the mixture was concentrated by rotary evaporation to dryness, and then dissolved in 1.4 mL of acetonitrile. The crude product was separated by preparative reverse liquid chromatography (separation conditions: Xtimate C18 150×40 mm×5 μm; mobile phase: [H₂O(HCl)-ACN]; ACN %: 30%-60%, 10 min) to obtain compound 4d.

MS (ESI, m/z): 506.0 [M+1]⁺.

### Step 5: Synthesis of Compound 4e

4d (0.05 g, 98.84 μmol) was weighed and dissolved in tetrahydrofuran (3 mL) and water (0.75 mL). Lithium hydroxide hydrate (20.74 mg, 494.19 μmol) was added at 0° C., and the mixture was stirred for 30 min, and then stirred at 20° C. for 16 hours. After the reaction was completed, the mixture was adjusted to pH of 3-4 with 2M hydrochloric acid, and concentrated by rotary evaporation to dryness to obtain the crude product 4e, which was directly used in the next reaction.

MS (ESI, m/z): 477.9 [M+1]+.

Step 6: Synthesis of Compound 4f

4e (0.04 g, 83.71 μmol) was weighed and dissolved in N,N-dimethylformamide (1 mL), followed by the addition of O-(7-azabenzotriazol-1-yl)-N,N,N′,N′-tetramethyluronium hexafluorophosphate (63.66 mg, 167.43 μmol), diisopropylethylamine (54.10 mg, 418.56 μmol, 72.91 μL), and ammonium chloride (13.43 mg, 251.14 μmol), and the mixture was stirred at 20° C. for 1 h. After the reaction was completed, ethyl acetate (10 mL) and water (10 mL) were added to the reaction solution. The layers were separated, and the aqueous phase was extracted twice with ethyl acetate (10 mL). The organic phases were combined and washed with saturated aqueous sodium bicarbonate solution (10 mL) and saturated brine (10 mL). The organic phase was concentrated by rotary evaporation to dryness. The crude product was separated by column chromatography (dichloromethane/methanol=1:0-20:1) to obtain 4f.

MS (ESI, m/z): 476.8 [M+1]+.

### Step 7: Synthesis of Compound 4

4f (0.01 g, 20.97 μmol) was weighed and dissolved in N,N-dimethylformamide dimethyl acetal (5.00 mL), and the mixture was stirred at 20° C. for 4 hours under nitrogen protection. Then the reaction solution was concentrated, dissolved in 1,2-dichloroethane (5 mL) and concentrated again. After concentration, the residue was dissolved in 1 mL of ethanol to prepare solution A. In addition, acetic acid (0.1 mL) was added to ethanol (0.5 mL), and methylhydrazine dihydrochloride (10.00 mg, 84.04 μmol) was added under an ice bath. Solution A was added dropwise. After the dropwise addition was completed, the mixture was stirred at 20° C. After the reaction was completed, the mixture was concentrated directly, and the crude product was separated by column chromatography (dichloromethane/methanol=1:0-20:1). The fraction containing the product was concentrated and lyophilized to obtain compound 4 (purity 78.90%).

MS (ESI, m/z): 514.9 [M+1]+.

¹H NMR (400 MHz, CDCl₃) δ=8.04-7.93 (m, 1H), 7.82 (s, 1H), 7.79-7.76 (m, 1H), 7.68-7.58 (m, 1H), 7.33 (s, 1H), 7.06 (s, 1H), 7.02-6.90 (m, 1H), 5.09-5.00 (m, 2H), 4.24-4.16 (m, 4H), 3.93-3.92 (m, 1H), 3.91-3.86 (m, 3H), 3.73 (s, 2H).

### Synthetic Route 2:

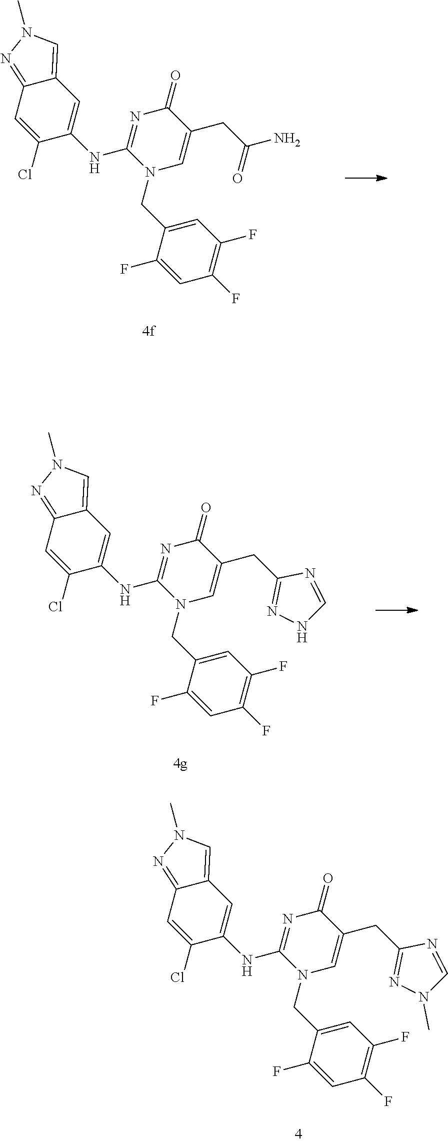 [SMILES: Cn1cc2cc(Nc3nc(=O)c(CC(N)=O)cn3Cc3cc(F)c(F)cc3F)c(Cl)cc2n1 ; Cn1cc2cc(Nc3nc(=O)c(Cc4nc[nH]n4)cn3Cc3cc(F)c(F)cc3F)c(Cl)cc2n1 ; Cn1cnc(Cc2cn(Cc3cc(F)c(F)cc3F)c(Nc3cc4cn(C)nc4cc3Cl)nc2=O)n1]

### Step 1: Synthesis of Compound 4g

4f (0.31 g, 650.12 μmol) was weighed and dissolved in N,N-dimethylformamide dimethyl acetal (5 mL), and the mixture was stirred at 20° C. for 4 hours under nitrogen protection. Then the reaction solution was concentrated, dissolved in 1,2-dichloroethane (5 mL) and concentrated again. After concentration, the residue was dissolved in 1 mL of ethanol to prepare solution A. In addition, acetic acid (0.7 mL) was added to ethanol (2 mL), and hydrazine hydrochloride (178.15 mg, 2.60 mmol) was added under and ice bath. Solution A was added dropwise. After the dropwise addition was completed, the mixture was stirred at 20° C. After the reaction was completed, the mixture was concentrated directly, and the crude product was separated by column chromatography (dichloromethane/methanol=1:0-20:1). The fraction containing the product was concentrated to obtain compound 4g.

MS (ESI, m/z): 500.9 [M+1]+.

¹H NMR (400 MHz, CDCl₃) δ=9.75 (br s, 1H), 7.85 (s, 1H), 7.79 (s, 1H), 7.69-7.57 (m, 1H), 7.44 (s, 1H), 7.10 (s, 1H), 6.99 (dt, J=6.5, 9.5 Hz, 1H), 6.71 (br s, 1H), 5.05 (s, 2H), 4.23 (s, 3H), 3.73 (s, 2H).

### Step 2: Synthesis of Compound 4 Hydrochloride

4g (130 mg, 259.55 μmol) was dissolved in N,N-dimethylformamide (3 mL). Potassium carbonate (71.74 mg, 519.10 μmol) was added, and iodomethane (44.21 mg, 311.46 μmol, 19.39 μL) was slowly added dropwise to the reaction solution. The reaction was stirred at 20° C. for 1 hour. After the reaction was completed, ethyl acetate (20 mL) and water (20 mL) were added to the reaction solution. The layers were separated, and the aqueous phase was extracted twice with ethyl acetate (20 mL). The organic phases were combined and washed with saturated brine (10 mL). The organic phase was concentrated, and the crude product was purified by preparative silica gel plate chromatography (dichloromethane/methanol=10:1), and then purified by preparative reverse liquid chromatography (separation conditions: Xtimate C18 150×40 mm×5 μm; mobile phase: [H₂O(HCl)-ACN]; ACN %: 17%-47%, 10 min). The fraction of the product was concentrated and lyophilized to obtain compound 4 hydrochloride (purity 98.4%).

MS (ESI, m/z): 515.2 [M+1]⁺.

¹H NMR (400 MHz, CDCl₃) δ=9.43 (s, 1H), 8.09 (br s, 2H), 7.74 (br s, 1H), 7.69 (s, 1H), 7.66-7.54 (m, 1H), 7.08-6.91 (m, 1H), 5.46 (br s, 2H), 4.15 (s, 3H), 3.96 (s, 3H), 3.25 (td, J=1.5, 3.1 Hz, 2H).

### Example 2

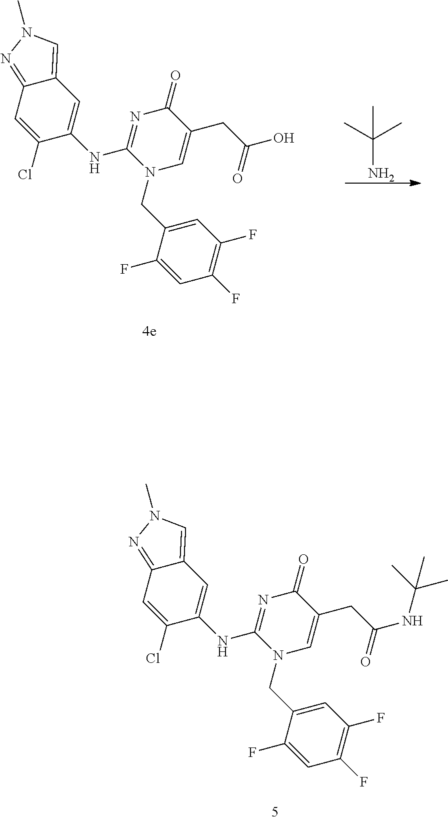

### Step 1: Synthesis of Compound 5

4e (0.025 g, 52.32 μmol) and tert-butylamine (11.48 mg) were weighed and dissolved in tetrahydrofuran (2 mL). Then 1-(3-dimethylaminopropyl)-3-ethylcarbodiimide (12.04 mg, 62.78 μmol), 1-hydroxybenzotriazole (8.48 mg, 62.78 μmol), and diisopropylethylamine (18.23 μL) were added. The mixture was stirred at 20° C. for 48 hours. After the reaction was completed, ethyl acetate (10 mL) and water (10 mL) were added to the reaction solution, and the layers were separated. The aqueous phase was extracted twice with ethyl acetate (10 mL), and the combined organic phases were washed with saturated sodium bicarbonate aqueous solution (10 mL) and saturated brine (10 mL). The organic phase was concentrated by rotary evaporation to dryness. The crude product was separated by column chromatography (petroleum ether/ethyl acetate=1:0-1:1) to obtain compound 5.

¹H NMR (400 MHz, CD₃OD) δ=8.12 (br s, 1H), 7.69 (s, 1H), 7.56 (s, 2H), 7.24 (br d, J=7.0 Hz, 1H), 5.18 (br s, 2H), 4.18 (s, 2H), 4.23-4.14 (m, 1H), 3.10 (s, 2H), 1.30 (s, 11H).

### Example 3

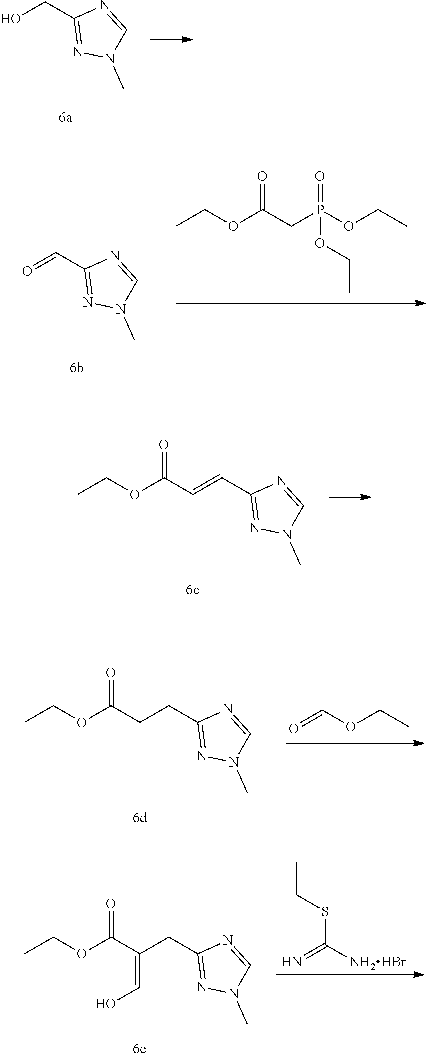

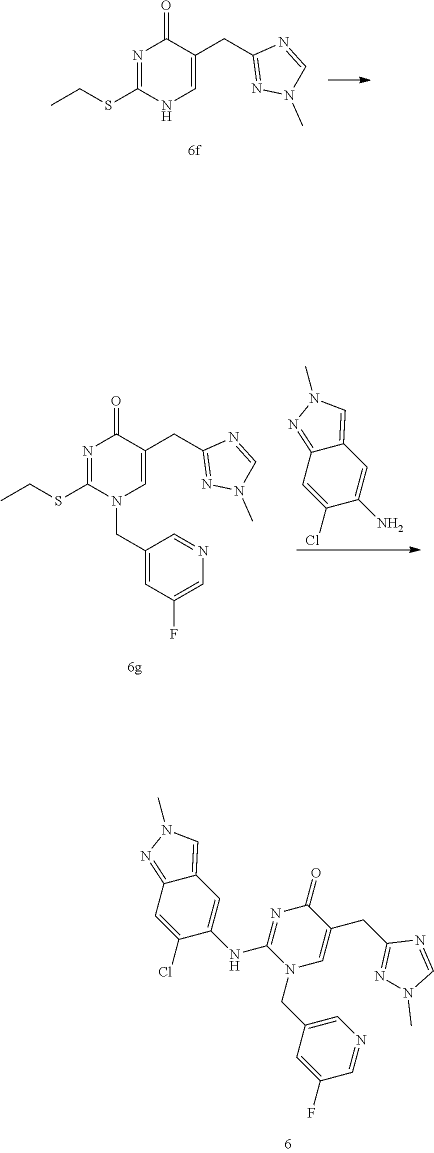 [SMILES: CCSc1nc(=O)c(Cc2ncn(C)n2)c[nH]1 ; CCSc1nc(=O)c(Cc2ncn(C)n2)cn1Cc1cncc(F)c1 ; Cn1cc2cc(N)c(Cl)cc2n1 ; Cn1cnc(Cc2cn(Cc3cncc(F)c3)c(Nc3cc4cn(C)nc4cc3Cl)nc2=O)n1]

### Step 1: Synthesis of Compound 6b

6a (5 g, 44.20 mmol) was dissolved in dichloromethane (50 mL). Diacetoxyiodobenzene (17.08 g, 53.04 mmol) was added, and then 2,2,6,6-tetramethylpiperidine oxide (695.09 mg, 4.42 mmol) was slowly added, and the reaction was stirred at 20° C. for 1 hour. The reaction solution was slowly added to water (50 mL), and extracted with dichloromethane (50 mL×2). The organic phases were combined and rotary-evaporated to dryness, and the crude product was purified by column chromatography (petroleum ether/ethyl acetate=1:1-0:1) to obtain 6b.

### Step 2: Synthesis of Compound 6c

6b (9.89 g, 44.10 mmol, 8.75 mL) was added to tetrahydrofuran (150 mL) and the reaction was stirred at 0° C. Potassium tert-butoxide (1 M, 52.92 mL) was then slowly added dropwise and the reaction was stirred at 0° C. for 0.5 hours. Triethyl phosphoacetate (4.9 g, 44.10 mmol) was added in batches and the reaction was stirred at 20° C. for 1 hour. Methanol (30 mL) was added to dissolve the solid and the crude product was purified by column chromatography (dichloromethane/methanol=20:1) to obtain 6c.

### Step 3: Synthesis of Compound 6d

6c (6.00 g, 35.87 mmol) was dissolved in a mixed solvent of dichloromethane and methanol (1:1, 100 mL). Palladium on carbon (10% content) was added under nitrogen protection, and the mixture was stirred at 20° C. for 12 hours in a hydrogen atmosphere (15 psi). The reaction solution was directly filtered, and the filter cake was washed with methanol (100 mL). The filtrate was rotary-evaporated to dryness to obtain 6d.

### Step 4: Synthesis of Compound 6e

Sodium hydride (2.13 g, 53.20 mmol, 60% content) was added to tetrahydrofuran (60 mL), and then ethanol (2.45 g, 53.20 mmol) was added. The reaction was stirred at 0° C. for 1 hour. Then, a solution of 6d (6 g, 35.47 mmol) in tetrahydrofuran (15 mL), and ethyl formate (2.89 g, 39.01 mmol, 3.14 mL) dissolved in tetrahydrofuran (15 mL) were slowly added dropwise. The reaction was stirred at 20° C. for 4 hours. The reaction solution was slowly added to ice water (60 mL), and extracted with methyl tert-butyl ether (60 mL×2). The aqueous phase containing 6e was directly used in the next step.

### Step 5: Synthesis of Compound 6f

The aqueous phase containing 6e and S-ethylisothiourea hydrobromide (6.13 g, 33.14 mmol) were added to water (200 mL), and sodium hydroxide (662.82 mg, 16.57 mmol) dissolved in water (200 mL) was slowly added. The reaction was stirred at 100° C. for 4 hours. The reaction solution was directly lyophilized, and the crude product was purified by column chromatography (dichloromethane/methanol=20:1) to obtain 6f.

¹H NMR (400 MHz, CD₃OD) δ=8.37-8.23 (m, 1H), 7.76 (br s, 1H), 3.89 (s, 3H), 3.81 (s, 2H), 3.19 (q, J=7.4 Hz, 2H), 1.38 (t, J=7.3 Hz, 3H).

### Step 6: Synthesis of Compound 6g

6e (50 mg, 198.96 μmol) was dissolved in acetonitrile (5 mL). 3-Bromomethyl-5-fluoropyridine hydrobromide (53.90 mg, 198.96 μmol) and potassium carbonate (82.49 mg, 596.88 μmol) were added, and the reaction was stirred at 60° C. for 2 hours. The reaction solution was directly filtered and the filtrate was rotary-evaporated to dryness. The crude product was purified by column chromatography (petroleum ether/ethyl acetate=5:1) to obtain 6g.

¹H NMR (400 MHz, CD₃OD) δ=8.49 (d, J=2.8 Hz, 1H), 8.43 (s, 1H), 8.34 (s, 1H), 7.86 (s, 1H), 7.68 (br d, J=9.1 Hz, 1H), 5.32 (s, 2H), 3.90 (s, 3H), 3.83 (s, 2H), 3.25 (q, J=7.4 Hz, 2H), 1.36 (t, J=7.3 Hz, 3H).

### Step 6: Synthesis of Compound 6 Hydrochloride

6g (19.57 mg, 54.29 μmol) and 6-chloro-2-methyl-2H-indazole-5-amine (11.83 mg, 65.15 μmol) were dissolved in tert-butyl alcohol (1 mL), and then acetic acid (75.00 mg) was added. The mixture was stirred at 130° C. for 12 hours under nitrogen protection. The reaction solution was cooled to room temperature, and a large amount of solid precipitated, which was directly filtered and the filter cake was washed with ethyl acetate (10 mL). The crude product was purified by preparative reverse liquid chromatography (separation conditions: Xtimate C18 150*40 mm*5 μm; mobile phase: [H₂O (HCl)-ACN]; B(ACN) %: 3%-33%, 10 min) to obtain compound 6 hydrochloride.

MS (ESI, m/z): 480.2 [M+1]+.

### Example 4

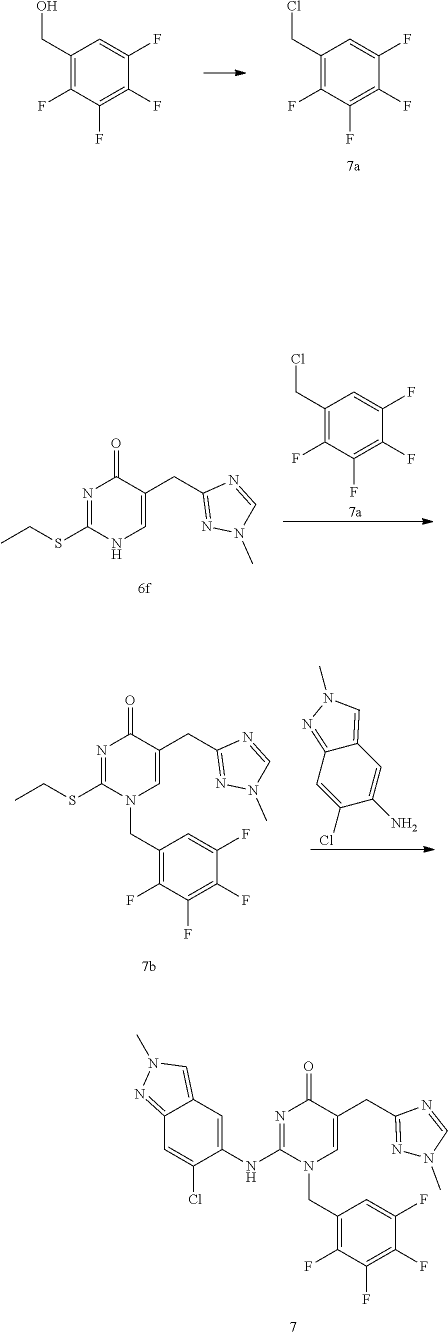

### Step 1: Synthesis of Compound 7a

(2,3,4,5-tetrafluorophenyl)methanol (0.16 g, 888.40 μmol) was dissolved in dichloromethane (3 mL), then thionyl chloride (317.08 mg, 2.67 mmol, 193.34 μL) and pyridine (7.03 mg, 88.84 μmol, 7.17 μL) were added. The mixture was reacted at 40° C. for 3 hours. After the reaction was completed, the reaction solution of 7a was directly used in the next step.

¹H NMR (400 MHz, CDCl₃) δ=7.16-7.02 (m, 1H), 4.58 (d, J=0.8 Hz, 2H).

### Step 2: Synthesis of Compound 7b

6f (0.2 g, 795.84 μmol) was dissolved in acetonitrile (4 mL), and then the above reaction solution of 7a and diisopropylethylamine (554.48 μL) were added. The mixture was reacted at 60° C. for 36 hours. The reaction solution was then concentrated to substantially dryness under reduced pressure, and then purified by preparative thin layer chromatography (Pre-TLC) (preliminary basification with triethylamine, DCM/MeOH=20:1, Rf=0.4) to obtain 7b.

MS (ESI, m/z): 413.9 [M+1].

¹H NMR (400 MHz, CD₃OD) δ=8.32 (s, 1H), 7.74 (s, 1H), 7.22-7.12 (m, 1H), 5.25 (s, 2H), 3.88 (s, 3H), 3.80 (s, 2H), 3.25-3.24 (m, 2H), 1.40-1.36 (m, 3H).

### Step 3: Synthesis of Compound 7

7b (0.1 g, 241.90 μmol) and 6-chloro-2-methyl-indazol-5-amine (65.90 mg, 362.85 μmol) were dissolved in glacial acetic acid (0.25 mL) and 2-methylbutan-2-ol (0.75 mL). The mixture was reacted at 130° C. for 36 hours under nitrogen protection. The reaction solution was then concentrated and the crude product was purified by preparative reverse liquid chromatography (separation conditions: Phenomenex C18 80×40 mm×3 μm; mobile phase: [H₂O(NH₃H₂O+NH₄HCO₃)-ACN]; B(ACN) %: 31%-61%, 8 min). The fraction of product was concentrated and lyophilized to obtain compound 7.

MS (ESI, m/z): 533.1 [M+1].

¹H NMR (400 MHz, CDCl₃) δ=7.99 (br s, 1H), 7.87-7.75 (m, 2H), 7.53-7.40 (m, 1H), 7.33 (br s, 1H), 7.05 (br s, 1H), 5.08 (br s, 2H), 4.21 (br s, 3H), 3.90 (br s, 3H), 3.82-3.67 (m, 2H).

¹⁹F NMR (377 MHz, CDCl₃) δ=−138.46 (s, 1F), −143.37 (s, 1F), −154.93 (s, 1F), −155.26 (s, 1F).

### Example 5

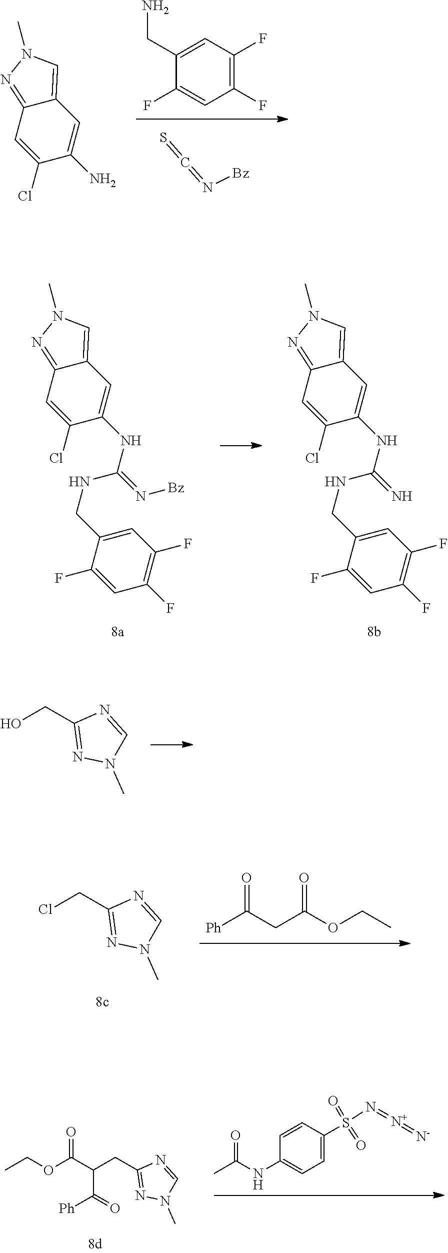

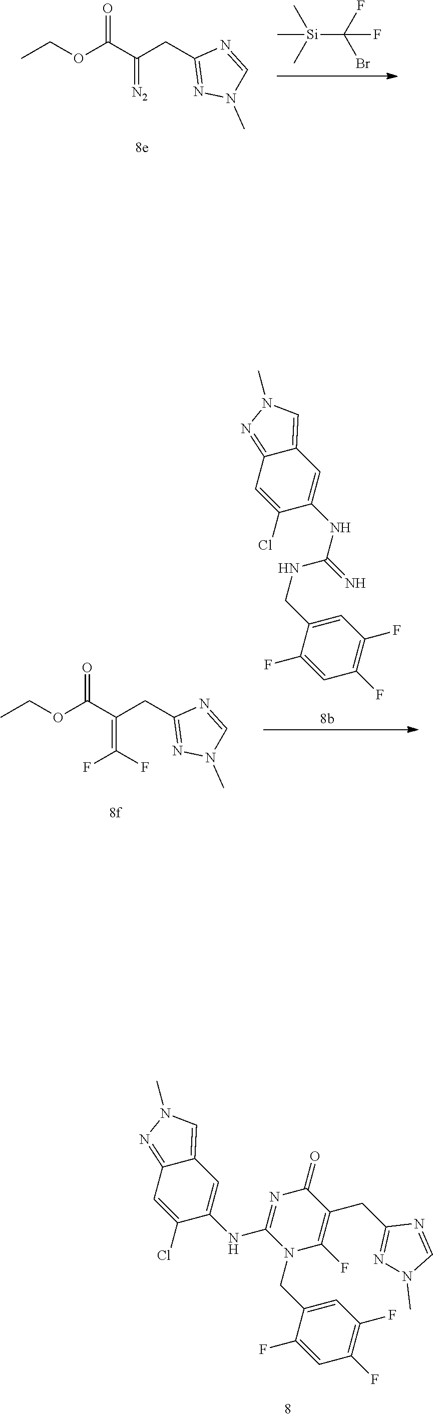

### Step 1: Synthesis of Compound 8a

6-chloro-2-methyl-2H-indazol-5-amine (16.91 g, 93.10 mmol) was dissolved in acetonitrile (450 mL), and benzoylisothiocyanate (5.06 g, 31.03 mmol, 4.19 mL) was added dropwise under an ice bath. After the dropwise addition was completed, the mixture was stirred at 20° C. for 1 hour. 2,4,5-Trifluorobenzylamine (22.50 g, 139.64 mmol), triethylamine (25.92 mL) and 1-(3-dimethylaminopropyl)-3-ethylcarbodiimide hydrochloride (21.68 g, 139.64 mmol) were slowly added to the reaction system in sequence under an ice bath, and the mixture was stirred at 20° C. for 16 hours. After the reaction was completed, the reaction system was concentrated to dryness under reduced pressure. Water (500 mL) was added to the reaction system. The mixture was extracted with dichloromethane (100 mL) three times, dried over anhydrous sodium sulfate, filtered, and concentrated under reduced pressure. The concentrate was slurried and stirred with 500 mL of a mixed solvent of petroleum ether and ethyl acetate (8:1) for 0.5 h. The mixture was filtered, and the solid was collected to obtain 8a, which was used directly in the next step.

MS (ESI, m/z): 472.1 [M+1]+.

### Step 2: Synthesis of Compound 8b

8a (64 g, 135.63 mmol) was dissolved in 1,4-dioxane (480 mL), and an aqueous solution (160 mL) of sodium hydroxide (27.13 g) was added. The mixture was stirred at 100° C. for 16 hours. After the reaction was completed, water (2000 mL) was added to the reaction system and a large amount of solid precipitated. The mixture was filtered by suction, and the filter cake was washed with water (100 mL). The filter cake was collected to obtain 8b, which was directly used in the next step reaction.

MS (ESI, m/z): 368.0 [M+1]+.

¹H NMR (400 MHz, CDCl₃): δ=7.79 (m, 1H), 7.75 (s, 1H), 7.45-7.55 (m, 3H), 7.25 (s, 2H), 6.89-6.95 (m, 1H), 4.56 (s, 2H), 4.18 (s, 3H).

### Step 3: Synthesis of Compound 8c

Under nitrogen protection, (1-methyl-1H-1,2,4-triazol-3-yl)methanol (20 g, 176.81 mmol) was dissolved in dichloromethane (1500 mL). The mixture was cooled to 0° C., and thionyl chloride (25.65 mL) was slowly added dropwise with stirring. After the dropwise addition was completed, the mixture was slowly warmed to 20° C. and stirred for 16 hours. After the reaction was completed, saturated sodium bicarbonate (400 mL) was added to adjust the pH to 7-8. The mixture was left to stand, and the layers were separated. The aqueous phase was extracted five times with dichloromethane:methanol=10:1 (200 mL). The organic phases were combined, dried over anhydrous sodium sulfate, filtered and concentrated under reduced pressure to obtain 8c, which was used directly in the next step.

MS (ESI, m/z): 131.9 [M+1]+.

¹H NMR (400 MHz, CDCl₃): δ=8.25 (m, 1H), 4.69 (s, 2H), 3.85 (s, 3H).

### Step 4: Synthesis of Compound 8d

To a reaction flask were added ethyl benzoyl acetate (27.19 g) and N,N-dimethylformamide (270 mL), followed by potassium carbonate (29.32 g), potassium iodide (2.35 g) and 8c (23.26 g) in sequence. The reaction solution was stirred at 40° C. for 16 hours. After the reaction was completed, the reaction solution was filtered, and the filtrate was concentrated in vacuum by an oil pump to remove most of the solvent. Ethyl acetate (200 mL) and water (100 mL) were added. The mixture was allowed to stand, and the layers were separated. The aqueous phase was extracted three times with ethyl acetate (200 mL), and the organic phases were combined and dried over anhydrous sodium sulfate, filtered, and concentrated under reduced pressure to obtain a crude product. The crude product was purified by column chromatography (petroleum ether/ethyl acetate=1:0−0:1) to obtain 8d.

MS (ESI, m/z): 288.0 [M+1]+.

### Step 5: Synthesis of Compound 8e

To a reaction flask were added 8d (24.45 g, 85.10 mmol), 4-acetylaminobenzenesulfonyl azide (30.67 g, 127.65 mmol) and acetonitrile (500 mL). A solution of 1.8-diazabicyclo[5.4.0]undecane-7-ene (19.43 g, 127.65 mmol) in acetonitrile (20 mL) was slowly added dropwise at 0° C. After the dropwise addition was completed, the reaction mixture was stirred at 25° C. for 16 hours. After the reaction was completed, water (600 mL) and dichloromethane (600 mL) were added to the reaction solution. The mixture was allowed to stand, and the layers were separated. The aqueous phase was extracted three times with dichloromethane (400 mL). The organic phases were combined and dried over anhydrous sodium sulfate, filtered and concentrated under reduced pressure to obtain the crude product. The crude product was purified by column chromatography (petroleum ether/ethyl acetate=1:0-2:3) to give 8e.

MS (ESI, m/z): 209.9 [M+1]⁺.

¹H NMR (400 MHz, CDCl₃): δ=7.95 (s, 1H), 4.23 (q, J=7.2 Hz, 2H), 3.87 (s, 3H), 3.75 (s, 2H), 1.26 (t, J=7.2 Hz, 3H).

### Step 6: Synthesis of Compound 8f

To a reaction flask were added 8e (7.0 g, purity: 74%) and 1,4-dioxane (210 mL), followed by (1-bromo-1,1-difluoro-methyl)trimethylsilane (12.57 g, 61.90 mmol) and tetrabutylammonium bromide (2.00 g). The atmosphere was replaced with nitrogen three times and the reaction solution was stirred at 120° C. for 1 hour. After the reaction was completed, the reaction solution was concentrated under reduced pressure to obtain a crude product. The crude product was dissolved in dichloromethane (40 mL) and then ethyl acetate (40 mL) was added. The mixture was then rapidly purified by an adsorption column (diatomaceous earth+silica gel+anhydrous sodium sulfate) [eluent: dichloromethane (800 mL), ethyl acetate (500 mL)]. The ethyl acetate fraction was concentrated to obtain a crude product of 8f, which was used directly in the next step.

MS (ESI, m/z): 231.9 [M+1]+.

### Step 7: Synthesis of Compound 8

To a reaction flask were added 8f (3.4 g, purity: 49%), 8b (2.43 g, 6.49 mmol) and N,N-dimethylformamide (70 mL), and then potassium carbonate (1.99 g) was added. The reaction solution was stirred at 40° C. for 1 hour. After the reaction was completed, the reaction solution was filtered through diatomaceous earth. The filter cake was washed with ethyl acetate (20 mL). Saturated ammonium chloride solution (20 mL) and water (130 mL) were added to the filtrate, and the mixture was allowed to stand, and the layers were separated. The aqueous phase was extracted four times with ethyl acetate (30 mL). The organic phases were combined, dried over anhydrous sodium sulfate, filtered and concentrated under reduced pressure. The residual N,N-dimethylformamide was removed by concentration on an oil pump under reduced pressure to obtain a crude product. The obtained crude product was separated by column chromatography (petroleum ether/ethyl acetate=1:0-0:1, then dichloromethane/methanol=1:0-10:1) to obtain a relatively pure crude product. The relatively pure crude product was further purified by preparative thin layer chromatography (Pre-TLC) (dichloromethane/methanol=20:1, developed twice) to obtain a purer crude product at the lower point. The purer crude product was purified by preparative reverse liquid chromatography (separation conditions: Waters Xbridge Prep OBD C18 150*40 mm*10 μm; mobile phase: [H₂O(NH₄HCO₃)-ACN]; B(ACN) %: 30%-50%, 8 min) to finally obtain compound 8.

MS (ESI, m/z): 533.0 [M+1]⁺.

¹H NMR (400 MHz, DMSO-d6): δ=10.30-10.60 (m, 1H), 8.28 (s, 1H), 8.20 (br s, 1H), 7.54-7.73 (m, 3H), 7.07-7.17 (m, 1H), 5.26 (br s, 2H), 4.12 (s, 3H), 3.77 (s, 3H), 3.57 (s, 2H).

### Example 6

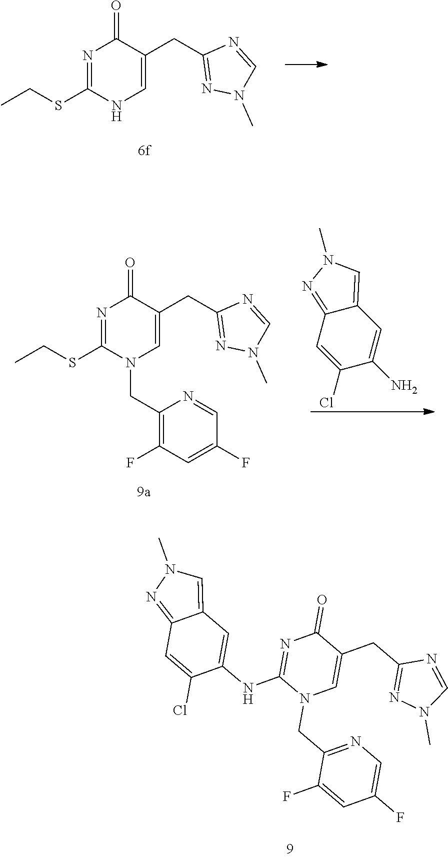 [SMILES: CCSc1nc(=O)c(Cc2ncn(C)n2)c[nH]1 ; CCSc1nc(=O)c(Cc2ncn(C)n2)cn1Cc1ncc(F)cc1F ; Cn1cc2cc(N)c(Cl)cc2n1 ; Cn1cnc(Cc2cn(Cc3ncc(F)cc3F)c(Nc3cc4cn(C)nc4cc3Cl)nc2=O)n1]

### Step 1: Synthesis of Compound 9a

6f (0.2 g, 795.84 μmol) was dissolved in acetonitrile (5 mL). 2-(Bromomethyl)-3,5-difluoropyridine hydrobromide (252.92 mg, 875.42 μmol) and N,N-diisopropylethylamine (415.85 μL) were added, and the reaction was stirred at 60° C. for 2 hours. The reaction solution was directly filtered and the filtrate was rotary-evaporated to dryness. The crude product was purified by column chromatography (dichloromethane/methanol=20:1) to obtain 9a.

¹H NMR (400 MHz, CD₃OD) δ=8.36 (d, J=2.3 Hz, 1H), 8.34-8.31 (m, 1H), 7.75 (s, 1H), 7.68 (ddd, J=2.5, 8.5, 9.8 Hz, 1H), 5.37 (s, 2H), 3.89 (s, 3H), 3.82 (s, 2H), 3.22-3.15 (m, 2H), 1.34-1.29 (m, 3H).

### Step 2: Synthesis of Compound 9 Hydrochloride

9a (0.1 g, 264.27 μmol) and 6-chloro-2-methyl-2H-indazol-5-amine (57.60 mg, 317.13 μmol) were dissolved in tert-butyl alcohol (3 mL), and then acetic acid (365.09 mg) was added. The mixture was stirred at 130° C. for 12 hours under nitrogen protection. The reaction solution was rotary-evaporated to dryness and the crude product was purified by preparative reverse liquid chromatography (separation conditions: Xtimate C18 150*40 mm*5 μm; mobile phase: [H₂O(HCl)-ACN]; B(ACN) %: 14%-34%, 10 min) to obtain compound 9 hydrochloride.

¹H NMR (400 MHz, DMSO-d6) δ=8.81 (s, 1H), 8.55 (s, 1H), 8.44 (s, 1H), 8.15-8.04 (m, 2H), 7.81 (s, 1H), 7.67 (br s, 1H), 5.69 (br s, 2H), 4.18 (s, 3H), 3.85 (s, 3H), 3.75 (br s, 2H).

### Example 7

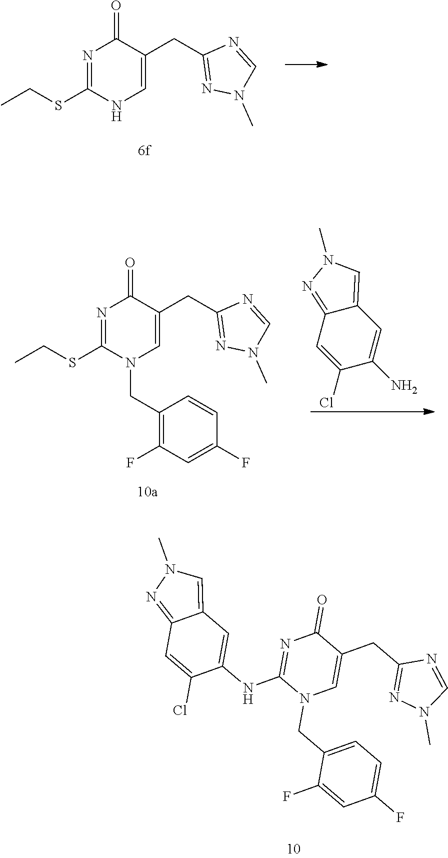 [SMILES: CCSc1nc(=O)c(Cc2ncn(C)n2)c[nH]1 ; CCSc1nc(=O)c(Cc2ncn(C)n2)cn1Cc1ccc(F)cc1F ; Cn1cc2cc(N)c(Cl)cc2n1 ; Cn1cnc(Cc2cn(Cc3ccc(F)cc3F)c(Nc3cc4cn(C)nc4cc3Cl)nc2=O)n1]

### Step 1: Synthesis of Compound 10a

6f (0.2 g, 795.84 μmol) was dissolved in acetonitrile (5 mL), and 2,4-difluorobenzyl bromide (181.23 mg, 875.42 μmol) and N,N-diisopropylethylamine (296.47 μL) were added. The reaction was stirred at 60° C. for 2 hours. The reaction solution was directly filtered and the filtrate was rotary-evaporated to dryness. The crude product was purified by column chromatography (petroleum ether/ethyl acetate=5:1) to obtain 10a.

¹H NMR (400 MHz, CD₃OD) δ=8.32 (s, 1H), 7.72 (s, 1H), 7.40-7.31 (m, 1H), 7.12-6.98 (m, 2H), 5.22 (s, 2H), 3.89 (s, 3H), 3.81 (s, 2H), 3.29-3.22 (m, 2H), 1.36 (s, 3H).

### Step 2: Synthesis of Compound 10 Hydrochloride

10a (300 mg, 794.89 μmol) and 6-chloro-2-methyl-2H-indazol-5-amine (173.24 mg, 953.87 μmol) were dissolved in tert-butyl alcohol (5 mL), and then acetic acid (1.10 g) was added. The mixture was stirred at 130° C. for 96 hours under nitrogen protection. The reaction solution was directly rotary-evaporated to dryness, and the crude product was purified by preparative reverse liquid chromatography (separation conditions: Xtimate C18 150*40 mm*5 μm; mobile phase: [H₂O(HCl)-ACN]; B(ACN) %: 24%-34%, 10 min) to obtain compound 10 hydrochloride.

¹H NMR (400 MHz, DMSO-d6) δ=8.80 (s, 1H), 8.43 (s, 1H), 8.04 (s, 1H), 7.80 (s, 1H), 7.65 (br s, 1H), 7.62-7.52 (m, 1H), 7.43-7.34 (m, 1H), 7.22-7.13 (m, 1H), 5.49 (br s, 2H), 4.18 (s, 3H), 3.85 (s, 3H), 3.73 (s, 2H).

### Example 8

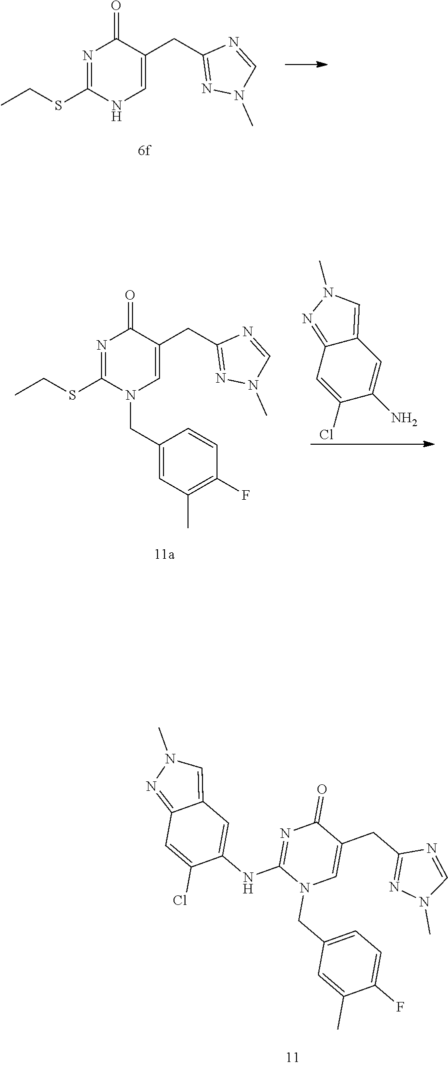 [SMILES: CCSc1nc(=O)c(Cc2ncn(C)n2)c[nH]1 ; CCSc1nc(=O)c(Cc2ncn(C)n2)cn1Cc1ccc(F)c(C)c1 ; Cn1cc2cc(N)c(Cl)cc2n1 ; Cc1cc(Cn2cc(Cc3ncn(C)n3)c(=O)nc2Nc2cc3cn(C)nc3cc2Cl)ccc1F]

### Step 1: Synthesis of Compound 11a

6f (0.2 g, 795.84 μmol) was dissolved in acetonitrile (5 mL). 4-Fluoro-3-methylbenzyl bromide (177.76 mg, 875.42 μmol) and N,N-diisopropylethylamine (296.47 μL) were added. The reaction was stirred at 60° C. for 2 hours. The reaction solution was directly rotary-evaporated to dryness, and the crude product was purified by column chromatography (dichloromethane/methanol=20:1) to obtain 11a.

¹H NMR (400 MHz, CD₃OD) δ=7.92 (s, 1H), 7.17 (s, 1H), 6.96-6.89 (m, 3H), 4.86 (s, 2H), 3.83-3.75 (m, 5H), 3.16 (q, J=7.4 Hz, 2H), 2.19 (d, J=2.0 Hz, 3H), 1.26 (t, J=7.4 Hz, 3H).

### Step 2: Synthesis of Compound 11 Hydrochloride

11a (140 mg, 374.89 μmol) and 6-chloro-2-methyl-2H-indazol-5-amine (81.71 mg, 449.86 μmol) were dissolved in tert-butyl alcohol (3 mL), and then acetic acid (1.11 g) was added. The mixture was stirred at 130° C. for 72 hours under nitrogen protection. The reaction solution was directly rotary-evaporated to dryness, and the crude product was purified by preparative reverse liquid chromatography (separation conditions: Welch Xtimate C18 100*40 mm*3 μm; mobile phase: [H₂O(HCl)-ACN]; B(ACN) %: 12%-42%, 8 min) to obtain compound 11 hydrochloride.

¹H NMR (400 MHz, CD₃OD) δ=9.38 (s, 1H), 8.40 (s, 1H), 8.30 (s, 1H), 7.92 (s, 1H), 7.83 (s, 1H), 7.41 (br d, J=6.9 Hz, 1H), 7.36-7.31 (m, 1H), 7.18 (t, J=9.0 Hz, 1H), 5.48 (s, 2H), 4.26 (s, 3H), 4.08 (s, 2H), 4.04 (s, 3H), 2.34 (d, J=1.5 Hz, 3H).

### Example 9

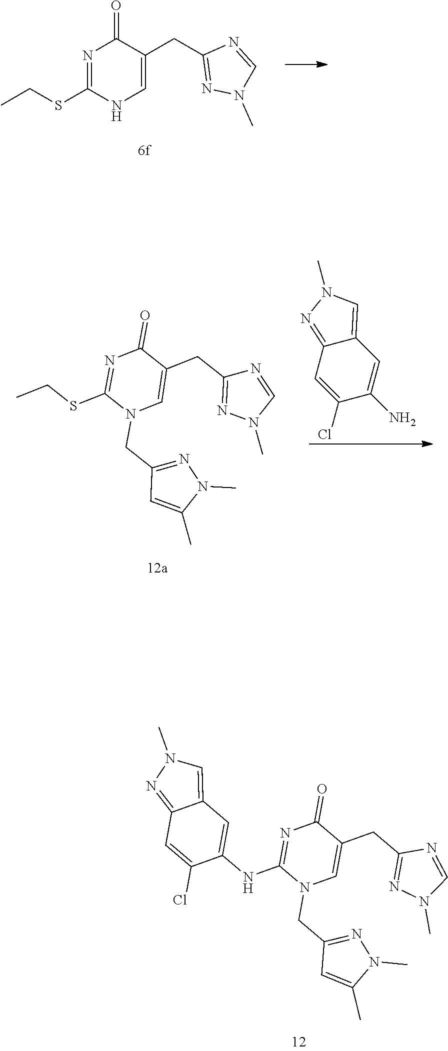

### Step 1: Synthesis of Compound 12a

6f (0.1 g, 397.92 μmol) was dissolved in acetonitrile (5 mL). 3-(Chloromethyl)-1,5-dimethyl-1H-pyrazole (115.08 mg, 795.84 μmol) and N,N-diisopropylethylamine (148.23 μL) were added. The reaction was stirred at 60° C. for 2 hours. The reaction solution was directly rotary-evaporated to dryness, and the crude product was purified by column chromatography (dichloromethane/methanol=20:1) to obtain 12a.

¹H NMR (400 MHz, CD₃OD) δ=8.40-8.26 (m, 1H), 7.67 (s, 1H), 6.10 (s, 1H), 5.05 (s, 2H), 3.89 (s, 3H), 3.79 (s, 2H), 3.76 (s, 3H), 3.25 (d, J=7.4 Hz, 2H), 2.33-2.24 (m, 3H), 1.39-1.36 (m, 3H).

### Step 2: Synthesis of Compound 12

12a (35 mg, 97.37 μmol) and 6-chloro-2-methyl-2H-indazol-5-amine (21.22 mg, 116.85 μmol) were dissolved in tert-butyl alcohol (3 mL), and then acetic acid (289.40 mg) was added. The mixture was stirred at 130° C. under nitrogen protection for 72 hours. The reaction solution was directly rotary-evaporated to dryness, and the crude product was purified by preparative reverse liquid chromatography (separation conditions: Xtimate C18 150*40 mm*5 μm; mobile phase: [H₂O(NH₃H₂O+NH₄HCO₃)-ACN]; B(ACN) %: 15%-35%, 24 min) to obtain compound 12.

¹H NMR (400 MHz, CD₃OD) δ=8.34-8.17 (m, 2H), 7.92 (br s, 1H), 7.73 (s, 1H), 7.54 (br s, 1H), 6.23 (s, 1H), 5.15-5.02 (m, 2H), 4.21 (s, 3H), 3.89 (s, 3H), 3.79 (s, 5H), 2.32 (s, 3H).

### Example 10

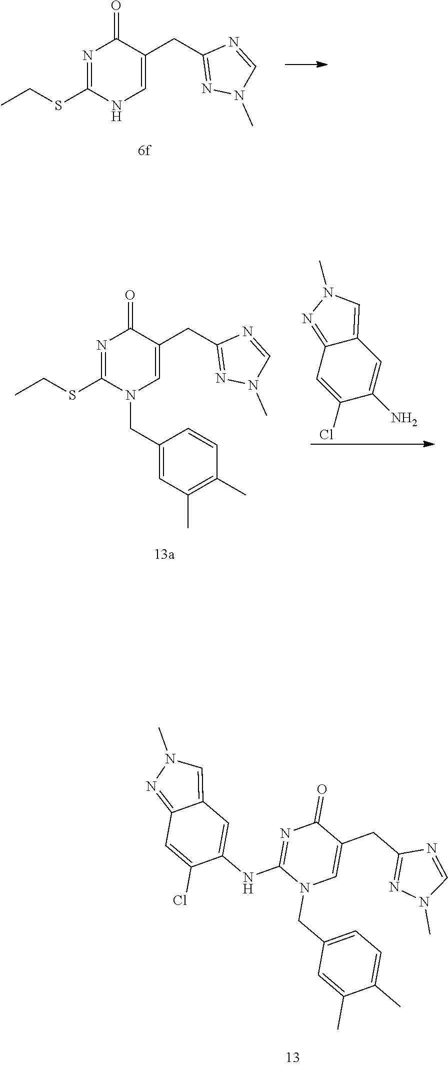 [SMILES: CCSc1nc(=O)c(Cc2ncn(C)n2)c[nH]1 ; CCSc1nc(=O)c(Cc2ncn(C)n2)cn1Cc1ccc(C)c(C)c1 ; Cn1cc2cc(N)c(Cl)cc2n1 ; Cc1ccc(Cn2cc(Cc3ncn(C)n3)c(=O)nc2Nc2cc3cn(C)nc3cc2Cl)cc1C]

### Step 1: Synthesis of Compound 13a

6f (0.2 g, 795.84 μmol) was dissolved in acetonitrile (5 mL). 3,4-Dimethylbenzyl bromide (158.44 mg, 795.84 μmol) and N,N-diisopropylethylamine (296.47 μL) were added. The reaction was stirred at 60° C. for 2 hours. The reaction solution was directly rotary-evaporated to dryness, and the crude product was purified by column chromatography (dichloromethane/methanol=20:1) to obtain 13a.

¹H NMR (400 MHz, CD₃OD) δ=8.35-8.25 (m, 1H), 7.66 (s, 1H), 7.15 (d, J=7.8 Hz, 1H), 7.05 (s, 1H), 7.02-6.97 (m, 1H), 5.09 (s, 2H), 3.87 (s, 3H), 3.79 (s, 2H), 3.23 (q, J=7.4 Hz, 2H), 2.27 (s, 6H), 1.36 (t, J=7.3 Hz, 3H).

### Step 2: Synthesis of Compound 13 Hydrochloride

13a (86.04 mg, 232.88 μmol) and 6-chloro-2-methyl-2H-indazol-5-amine (50.75 mg, 279.45 μmol) were dissolved in tert-butyl alcohol (5 mL), and then acetic acid (874.69 mg) was added. The mixture was stirred at 130° C. for 72 hours under nitrogen protection. The reaction solution was directly rotary-evaporated to dryness, and the crude product was purified by preparative reverse liquid chromatography (separation conditions: Welch Xtimate C18 100*40 mm*3 μm; mobile phase: [H₂O(HCl)-ACN]; B(ACN) %: 12%-42%, 8 min) to obtain compound 13 hydrochloride.

¹H NMR (400 MHz, CD₃OD) δ=9.57 (s, 1H), 8.42 (s, 1H), 8.31 (s, 1H), 7.95 (s, 1H), 7.83 (s, 1H), 7.32-7.18 (m, 3H), 5.47 (s, 2H), 4.26 (s, 3H), 4.16-4.01 (m, 5H), 2.33 (d, J=6.5 Hz, 6H).

### Example 11

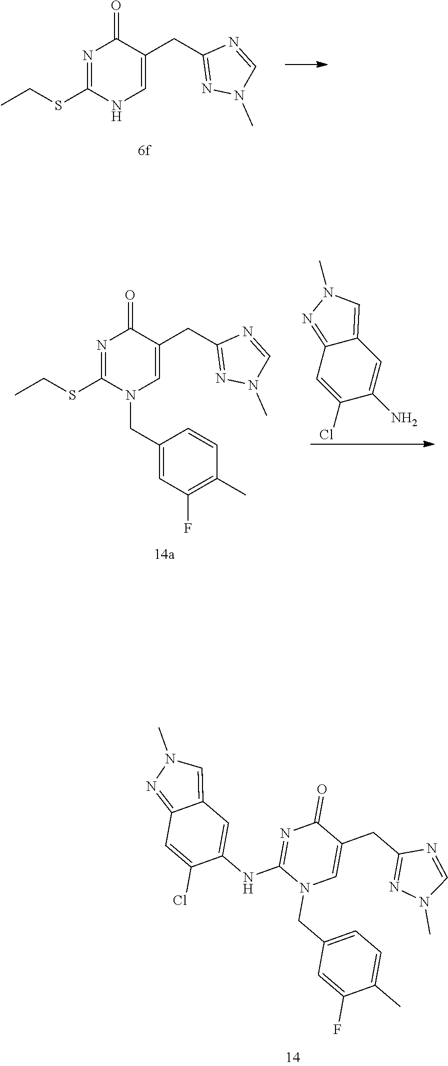 [SMILES: CCSc1nc(=O)c(Cc2ncn(C)n2)c[nH]1 ; CCSc1nc(=O)c(Cc2ncn(C)n2)cn1Cc1ccc(C)c(F)c1 ; Cn1cc2cc(N)c(Cl)cc2n1 ; Cc1ccc(Cn2cc(Cc3ncn(C)n3)c(=O)nc2Nc2cc3cn(C)nc3cc2Cl)cc1F]

### Step 1: Synthesis of Compound 14a

6f (0.2 g, 795.84 μmol) was dissolved in acetonitrile (5 mL). 3-Fluoro-4-methylbenzyl bromide (161.60 mg, 795.84 μmol) and N,N-diisopropylethylamine (296.47 μL) were added. The reaction was stirred at 60° C. for 2 hours. The reaction solution was directly rotary-evaporated to dryness, and the crude product was purified by column chromatography (dichloromethane/methanol=20:1) to obtain 14a.

¹H NMR (400 MHz, CD₃OD) δ=8.32 (s, 1H), 7.74 (s, 1H), 7.28 (t, J=7.8 Hz, 1H), 7.01 (d, J=9.0 Hz, 2H), 5.16 (s, 2H), 3.89 (s, 3H), 3.81 (s, 2H), 3.28-3.20 (m, 2H), 2.28 (d, J=1.4 Hz, 3H), 1.38-1.35 (m, 3H).

### Step 2: Synthesis of Compound 14

14a (90 mg, 241.00 μmol) and 6-chloro-2-methyl-2H-indazol-5-amine (52.52 mg, 289.20 μmol) were dissolved in tert-butyl alcohol (5 mL), and then acetic acid (905.19 mg) was added. The mixture was stirred at 130° C. for 72 hours under nitrogen protection. The reaction solution was directly rotary-evaporated to dryness, and the crude product was purified by preparative reverse liquid chromatography (separation conditions: Xtimate C18 150*40 mm*5 μm; mobile phase: [H₂O(NH₃·H₂O+NH₄HCO₃)-ACN]; B(ACN) %: 26%-46%, 24 min) to obtain 14.

¹H NMR (400 MHz, CD₃OD) δ=8.29 (s, 1H), 8.15 (br s, 1H), 7.76-7.67 (m, 1H), 7.53 (br s, 1H), 7.28 (br t, J=7.6 Hz, 1H), 7.15 (br s, 2H), 6.27-6.25 (m, 1H), 5.25-5.12 (m, 2H), 4.19 (s, 3H), 3.87 (s, 3H), 3.71 (br s, 2H), 2.29 (d, J=0.9 Hz, 3H).

### Example 12

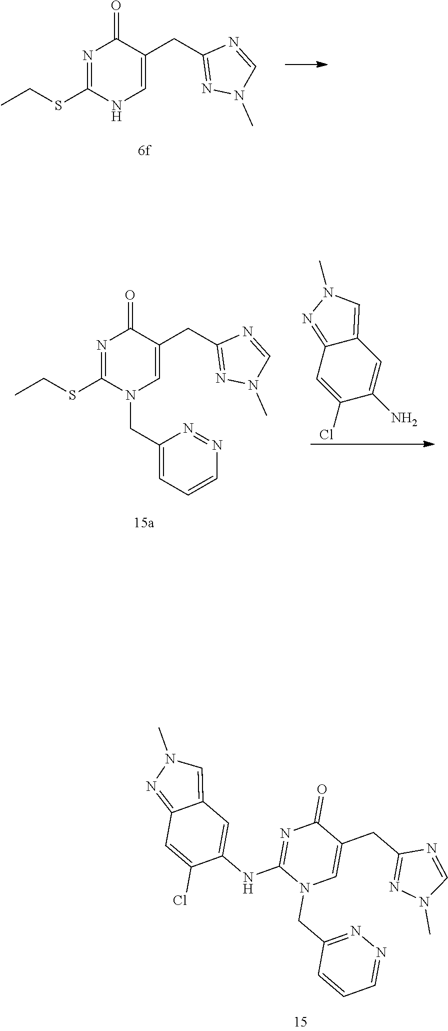 [SMILES: CCSc1nc(=O)c(Cc2ncn(C)n2)c[nH]1 ; CCSc1nc(=O)c(Cc2ncn(C)n2)cn1Cc1cccnn1 ; Cn1cc2cc(N)c(Cl)cc2n1 ; Cn1cnc(Cc2cn(Cc3cccnn3)c(Nc3cc4cn(C)nc4cc3Cl)nc2=O)n1]

### Step 1: Synthesis of Compound 15a

6f (0.2 g, 795.84 μmol) was dissolved in acetonitrile (5 mL). 3-(Bromomethyl)pyridazine hydrobromide (303.12 mg, 1.19 mmol) and N,N-diisopropylethylamine (415.85 μL) were added. The reaction was stirred at 60° C. for 2 hours. The reaction solution was directly rotary-evaporated to dryness, and the crude product was purified by column chromatography (dichloromethane/methanol=20:1) to obtain 15a.

¹H NMR (400 MHz, CD₃OD) δ=9.18 (dd, J=2.2, 4.3 Hz, 1H), 8.32 (s, 1H), 7.85 (s, 1H), 7.80-7.76 (m, 2H), 5.54 (s, 2H), 3.89 (s, 3H), 3.83 (s, 2H), 3.27-3.18 (m, 2H), 1.32 (t, J=7.3 Hz, 3H).

### Step 2: Synthesis of Compound 15

15a (105.55 mg, 307.35 μmol) and 6-chloro-2-methyl-2H-indazol-5-amine (66.99 mg, 368.82 μmol) were dissolved in tert-butyl alcohol (5 mL), and then acetic acid (1.15 g) was added. The mixture was stirred at 130° C. for 72 hours under nitrogen protection. The reaction solution was directly rotary-evaporated to dryness, and the crude product was purified by preparative reverse liquid chromatography (separation conditions: Xtimate C18 150*40 mm*5 μm; mobile phase: [H₂O(NH₃·H₂O+NH₄HCO₃)-ACN]; B(ACN) %: 14%-27%, 16 min) to obtain compound 15.

¹H NMR (400 MHz, CD₃OD) δ=9.16 (br s, 1H), 8.31 (s, 1H), 8.10 (br s, 1H), 7.92 (br d, J=7.8 Hz, 1H), 7.83-7.59 (m, 3H), 7.13 (br s, 1H), 5.43 (br s, 2H), 4.18 (s, 3H), 3.89 (s, 3H), 3.72 (br s, 2H).

### Example 13

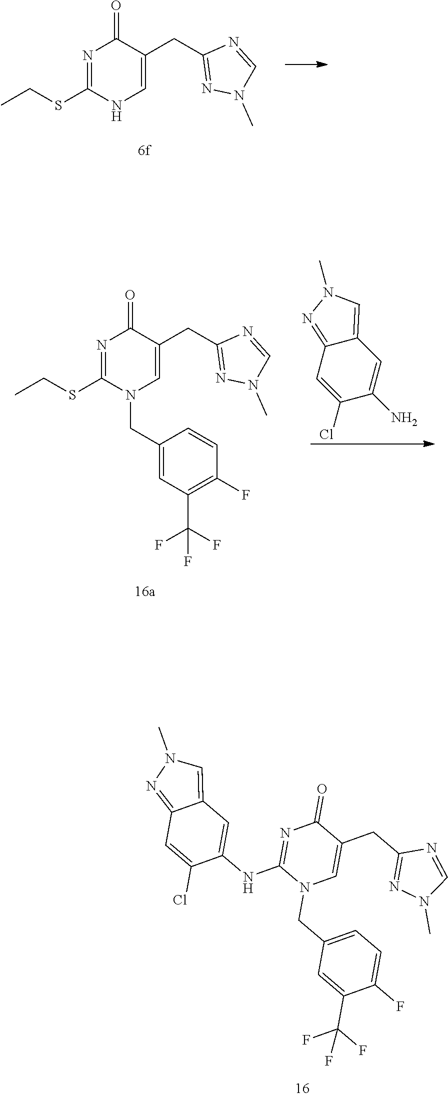 [SMILES: CCSc1nc(=O)c(Cc2ncn(C)n2)c[nH]1 ; CCSc1nc(=O)c(Cc2ncn(C)n2)cn1Cc1ccc(F)c(C(F)(F)F)c1 ; Cn1cc2cc(N)c(Cl)cc2n1 ; Cn1cnc(Cc2cn(Cc3ccc(F)c(C(F)(F)F)c3)c(Nc3cc4cn(C)nc4cc3Cl)nc2=O)n1]

### Step 1: Synthesis of Compound 16a

6f (0.2 g, 795.84 μmol) was dissolved in acetonitrile (5 mL). 4-Fluoro-3-trifluoromethylbenzyl bromide (245.46 mg, 955.01 μmol) and N,N-diisopropylethylamine (296.47 μL) were added. The reaction was stirred at 60° C. for 2 hours. The reaction solution was directly rotary-evaporated to dryness, and the crude product was purified by column chromatography (dichloromethane/methanol=20:1) to obtain 16a.

### Step 2: Synthesis of Compound 16

16a (90 mg, 210.57 μmol) and 6-chloro-2-methyl-2H-indazol-5-amine (45.89 mg, 252.68 μmol) were dissolved in tert-butyl alcohol (5 mL), and then acetic acid (790.89 mg) was added. The mixture was stirred at 130° C. under nitrogen protection for 72 hours. The reaction solution was directly rotary-evaporated to dryness, and the crude product was purified by preparative reverse liquid chromatography (separation conditions: Xtimate C18 150*40 mm*5 μm; mobile phase: [H₂O(NH₃·H₂O+NH₄HCO₃)-ACN]; B(ACN) %: 30%-50%, 24 min) to obtain compound 16.

¹H NMR (400 MHz, CDCl₃) δ=7.99 (s, 1H), 7.83 (s, 1H), 7.80 (s, 1H), 7.72 (br d, J=6.5 Hz, 2H), 7.33 (s, 1H), 7.26-7.20 (m, 1H), 7.24 (t, J=9.6 Hz, 1H), 7.08 (s, 1H), 5.13 (s, 2H), 4.22 (s, 3H), 3.90 (s, 3H), 3.76 (s, 2H).

### Example 14

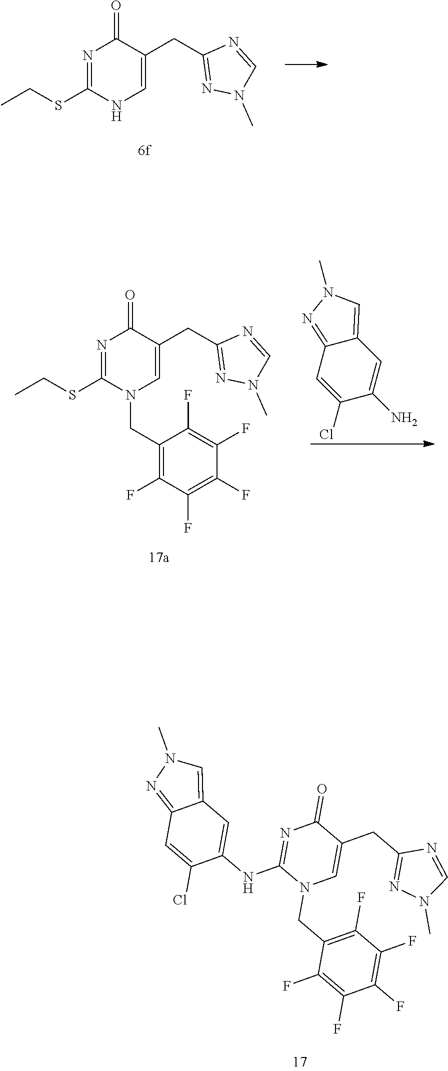 [SMILES: CCSc1nc(=O)c(Cc2ncn(C)n2)c[nH]1 ; CCSc1nc(=O)c(Cc2ncn(C)n2)cn1Cc1c(F)c(F)c(F)c(F)c1F ; Cn1cc2cc(N)c(Cl)cc2n1 ; Cn1cnc(Cc2cn(Cc3c(F)c(F)c(F)c(F)c3F)c(Nc3cc4cn(C)nc4cc3Cl)nc2=O)n1]

### Step 1: Synthesis of Compound 17a

6f (0.2 g, 795.84 μmol) was dissolved in a mixed solvent of water and acetonitrile (1:1, 2 mL). 2,3,4,5,6-Pentafluorobenzyl bromide (228.47 mg, 875.42 μmol) and N,N-diisopropylethylamine (277.24 μL) were added. The reaction was stirred at 60° C. for 2 hours. The reaction solution was directly added to water (20 mL). The mixture was extracted with ethyl acetate (20 mL×3), and the organic phase was rotary-evaporated to dryness. The crude product was purified by column chromatography (dichloromethane/methanol=20:1) to obtain 17a.

¹H NMR (400 MHz, CD₃OD) δ=8.34 (s, 1H), 7.76 (s, 1H), 5.36 (s, 2H), 3.90 (s, 3H), 3.80 (s, 2H), 3.26 (q, J=7.4 Hz, 2H), 1.37 (t, J=7.3 Hz, 3H).

### Step 2: Synthesis of Compound 17

17a (0.15 g, 347.72 μmol) and 6-chloro-2-methyl-2H-indazol-5-amine (75.78 mg, 417.26 μmol) were dissolved in tert-butyl alcohol (2 mL), and then acetic acid (696.02 μL) was added.

The mixture was stirred at 100° C. for 24 hours under nitrogen protection. The reaction solution was directly rotary-evaporated to dryness, and the crude product was purified by preparative reverse HPLC (separation conditions: Xtimate C18 150*40 mm*5 μm; mobile phase: [H₂O(NH₃—H₂O+NH₄HCO₃)-ACN]; B(ACN) %: 26%-46%, 24 min) to obtain compound 17.

¹H NMR (400 MHz, CDCl₃) δ=7.98 (s, 1H), 7.76 (br d, J=5.27 Hz, 2H), 7.36 (s, 1H), 7.01 (s, 1H), 5.09 (s, 2H), 4.19 (s, 3H), 3.90 (s, 3H), 3.74 (s, 2H).

### Biological Assay:

### Assay Example 1: Evaluation of the In Vitro Anti-Novel Coronavirus Mpro Protease Activity of the Assay Compounds

### 1. Assay Materials:

### 1.1 Reagents and Consumables are Shown in Table 1 Above.

### 1.2 Instruments are Shown in Table 2 Above.

### 2. Assay Method:

The compound was dissolved in DMSO, and diluted in a 3-fold gradient with Echo655 according to the concentration requirements for the assay to 10 concentration points. Duplicate assays were set at each concentration. The diluted solution was added to a 384-well plate. Mpro protein and substrate were diluted with assay buffer (100 mM NaCl, 20 mM Tris-HCl, 1 mM EDTA), and Mpro protein was added to the 384-well assay plate, incubated with the compound for 30 min at room temperature. Then the substrate was added. The assay concentration of Mpro protein was 25 nM, and the assay concentration of substrate was 25 μM. After incubating for 60 minutes in a 30° C. constant temperature incubator, the fluorescence signal value of Ex/Em=340 nm/490 nm was detected by microplate reader. At the same time, the background well containing the substrate and compound but not containing Mpro protein was detected as control.

### 3. Data Analysis:

1) The inhibition rate was calculated using the following formula:

$$Inhibition rate \% = [ ( Compound - {BG}_{Compound} ) - ( ZPE - {BG}_{ZPE )} ] /$$

$$[ ( HPE - {BG}_{HPE )} - ( ZPE - {BG}_{ZPE )} ] * 100 \%$$

^{#}HPE: 100% inhibition control, containing 25 nM Mpro protein+25 μM substrate+1 μM GC376
ZPE: No-inhibition control, containing 25 nM Mpro protein+25 μM substrate, not containing compound
Compound: Assay compound well, containing 25 nM Mpro protein+25 μM substrate+compound
BG: Background control well, containing 25 μM substrate+compound, not containing Mpro protein

2) Log (agonist) vs. response—variable slope nonlinear fitting analysis was carried out on the inhibition rate data (inhibition rate %) of the compound by using GraphPad Prism software, and the IC₅₀ value of the compound was obtained.

The inhibitory activity of the compounds of the present disclosure against the novel coronavirus Mpro protease is shown in Table 6.

TABLE 6
Inhibitory activity of the compounds of the present disclosure
against the novel coronavirus Mpro protease

| Compound No. | IC₅₀ (nM) |
|---|---|
| 4 | 25 |
| 7 | 62 |
| 8 | 58 |

Conclusion: The compounds of the present disclosure have better in vitro anti-novel coronavirus Mpro protease activity.

### Assay Example 2: Evaluation of In Vitro Antiviral Activity of Compounds Using a Novel Coronavirus Replicon System

### 1. Assay Materials

### 1.1 Cells

Huh7 cells were obtained from JCRB cell bank and cultured in DMEM supplemented with 10% FBS, 1% L-glutamine, 1% NEAA, and 1% double antibody.

### 1.2 Main Reagents and Instruments

Main detection reagents: luminescent cell viability assay kit CellTiter Glo (Promega).

Main equipment: Acumen Cellista (TTP LabTech).

### 2. Assay Method

TABLE 7
Assay method for antiviral activity

| | | Time for compound | Positive |
|---|---|---|---|
| Virus | Cell | treatment (days)/assay | Control |
| SARS-CoV-2 | Huh7 | 1/GFP | GC376 |
| replicon | | | |

On the first day, the compound was diluted in multiples and 0.3 μL per well was added to a 384-well microplate. After the SARS-CoV-2 replicon RNA was electrotransfected into Huh7 cells, 60 μL of cells was inoculated into the microplate containing the compound diluted in multiples at a density of 4000 cells/well. At the same time, a ZPE control (cells electrotransfected with SARS-CoV-2 replicons, without compound treatment) and an HPE control (culture medium control) were set up. The final concentration of DMSO in the culture medium was 0.5%, and the cells were cultured in a 5% CO₂, 37° C. incubator for 1 day.

On the second day, the number of GFP-expressing cells in each well was detected using an Acumen instrument, and the data were used for antiviral activity analysis. The cytotoxicity assay was carried out under the same conditions as the antiviral assay. On the second day, the cell viability detection reagent CellTiter Glo was added in a dark environment, and the cell viability of each well was detected using a BioTek microplate reader. The data were used for sample cytotoxicity analysis.

### Data Analysis

GraphPad Prism software (four parameter logistic equations) was used to perform nonlinear fitting analysis on the antiviral activity and cell viability of the samples, and to calculate the half effective concentration (EC₅₀) and half cytotoxic concentration (CC₅₀) values of the samples.

The antiviral activity and cytotoxicity of the compound were expressed by the inhibition rate (%) and cell viability (%) of the compound with different concentrations against SARS-COV-2 Replicon, respectively. The calculation formula is as follows:

$$Inhibition rate ( \% ) = ( GFP reading of assay well - average value of ZPE ) / ( average value of HPE control - average value of ZPE control ) \times 100$$

$$Cell viability ( \% ) = ( CTG reading of assay well - average value of HPE control ) / ( average value of ZPE control - average value of HPE control ) \times 100$$

The in vitro antiviral activity of the compounds of the present disclosure at the cellular level in the novel coronavirus replicon system is shown in Table 8.

TABLE 8
Antiviral activity of the compounds of the present disclosure at the
cellular level in vitro in the novel coronavirus replicon system

| Compound No. | EC₅₀ (nM) |
|---|---|
| 4 | 66 |
| 7 | 82 |
| 8 | 58 |

Conclusion: The compounds of the present disclosure have better in vitro antiviral activity at the cellular level.

### Assay Example 3: Assay of In Vitro Anti-Novel Coronavirus Activity

### 1. Assay Materials

### 1.1 Cells

African green monkey kidney (Vero) cells were obtained from the American Type Culture Collection (ATCC) and cultured in Dulbecco's Modified Eagle's Medium (DMEM, WelGene) supplemented with 10% fetal bovine serum (Gibco) and 1% double antibody (Gibco).

### 1.2 Viruses

The novel coronavirus βCoV/KOR/KCDC03/2020 strain was provided by the Korea Centers for Disease Control and Prevention (KCDC), sequence number NCCP43326.

### 2. Assay Method

Vero cells were trypsinized and diluted to 480,000 cells per ml in assay medium. The diluted cells were added to a 384-well cell assay plate using an automatic dispenser, 25 μL per well, 12,000 cells. The cells were cultured overnight in a 5% CO₂, 37° C. incubator.

On the second day, the compound and CP-100356 were diluted with DMSO, and then the diluted compound was added to the assay cell well using a liquid workstation. Then 25 μL of SARS-CoV-2 virus diluted in the assay culture medium was added to each well, MOI=0.0125. Cell controls (cells, without compound treatment or virus infection) and no compound treatment controls (cells infected with virus, without compound treatment, 0.5% DMSO added), as well as CP-100356 control (cells infected with virus, 2 μM CP-100356 treatment) were set up. The final volume of cell culture medium in each well was 50 μL. The cells were cultured in a 5% CO₂, 37° C. incubator for 24 hours.

At 24 hours after virus infection, 17 μL of 16% paraformaldehyde was added to each well. Then, the plate was placed at room temperature for 30 minutes; the supernatant was removed and the plate was washed twice with DPBS; 25 μL of 0.25% TritonX-100 was added to each well and the plate was placed at room temperature for 20 minutes; 0.25% TritonX-100 was removed and the plate was washed twice with DPBS; 25 μL of diluted primary antibody (1:3000 dilution) was added to each well and the plate was incubated at 37° C. for 1 hour; the primary antibody was removed and the plate was washed twice with DPBS; 25 μL of diluted secondary antibody Alexa Fluor 488-labeled goat anti-rabbit IgG (1:2000 dilution) and 2.5 μg/mL (1:4000 dilution) Hoechst 33342 were added to each well and the plate was incubated at 37° C. for 1 hour; the secondary antibody and Hoechst were removed and the plate was washed twice with DPBS; the plate was read using the high-content imaging analyzer Operetta, instrument settings: 488/405 emission, 20× objective, 5 fields of view per well.

### 3. Data Analysis

Columbus software was used to quantitatively analyze the total number of cells (number of cells stained with Hoechst) and the number of cells infected with the novel coronavirus (number of cells labeled with Alexa Fluor 488) in the images obtained by reading the plate with the high-content imaging analyzer. The data of infected cell ratio and total cell number were used for the analysis of the antiviral activity and cytotoxicity of the compound. The calculation formula is as follows:

$$Inhibition rate ( \% ) = 100 - ( infected cell ratio in assay well - average infected cell ratio in cell control well ) / ( average infected cell ratio in control well without compound treatment - average infected cell ratio in cell control well ) \times 100$$

$$Cell viability ( \% ) = total number of cells in the assay well /$$

$$average total number of cells in$$

$$the control well without compound treatment \times 100$$

XLfit 4 software was used to perform nonlinear fitting analysis on the inhibitory activity and cell viability of the compound and to calculate the IC50 and CC50 values of the compound. The fitting method was “Sigmoidal dose-response”. The calculation formula for IC₅₀ and CC₅₀ was: Y=Bottom+(Top Bottom)/(1+(IC50/X)Hillslope).

The in vitro antiviral activity of the compounds of the present disclosure in the novel coronavirus replicon system at the cellular level is shown in Table 9.

TABLE 9
In vitro activity of the compounds of the present
disclosure against the novel coronavirus

| Compound No. | EC₅₀ (nM) |
|---|---|
| Compound 4 | 52 |
| hydrochloride | |

Conclusion: The compounds of the present disclosure have better in vitro anti-novel coronavirus activity at the cellular level.

### Assay Example 4: Determination of Plasma Protein Binding Rate (PPB)

597 μL of blank plasma from CD-1 mice, SD rats, beagle dogs, cynomolgus monkeys and humans were weighed, and the working solution of the assay compound or the working solution of warfarin was added, so that the final concentration of the assay compound and warfarin in the plasma sample was 2 μM. The samples were mixed thoroughly. The final concentration of DMSO as an organic phase was 0.5%; 50 μL of the assay compound and warfarin plasma samples were pipetted into a sample receiving plate, and the corresponding volume of blank plasma or buffer was immediately added, so that the final volume of each sample well was 100 μL, and the volume ratio of plasma:dialysis buffer was 1:1, and then the stop solution was added to these samples. The samples were used as the To sample for recovery and stability determination. The assay compound and warfarin plasma samples were added to the dosing end of each dialysis well, and blank dialysis buffer was added to the receiving end corresponding to the dialysis well. Then the dialysis plate was sealed with a gas permeable membrane and placed in a humidified 5% CO2 incubator, and incubated with shaking at 37° C. and 100 rpm for 4 hours. After dialysis was completed, 50 μL of the dialyzed buffer sample and the dialyzed plasma sample were transferred to a new sample receiving plate. The corresponding volume of blank plasma or buffer was added to the sample so that the final volume of each sample well was 100 μL, and the volume ratio of plasma:dialysis buffer was 1:1. After protein precipitation, all samples were analyzed by LC/MS/MS, and the free rate (% Unbound) of the compound was calculated by the following formula:

$$\% Unbound = 100 * {F}_{C} / {T}_{C}$$

where F_{C} is the concentration of the compound at the buffer end of the dialysis plate; T_{C} is the concentration of the compound at the plasma end of the dialysis plate; and T₀ is the concentration of the compound in the plasma sample at time zero.

The assay results are shown in Table 10.

TABLE 10
Results of plasma protein binding assay

| | PPB_Unbound (%) | | | | |
|---|---|---|---|---|---|
| | CD-1 | SD | | Cynomolgus | |
| Compound | mice | rats | Beagle | monkey | Human |
| Compound 4 | 2.8 | 4.5 | 23.9 | 18.8 | 24.3 |
| hydrochloride | | | | | |

Assay conclusion: The compound of the present disclosure exhibits a high degree of binding rate in the plasma of CD-1 mice and SD rats, and a moderate degree of binding rate in the plasma of beagle dogs, cynomolgus monkeys and humans.

### Assay Example 5: Pharmacokinetic (PK) Assay

Objective of assay: The druggability of compounds was evaluated by measuring their pharmacokinetic properties in different animal species.

Assay Materials: C57BL6/J strain mice, Sprague-Dawley strain rats and beagle dogs.

### Assay Procedure:

The pharmacokinetic characteristics of the compound after intravenous and oral administration were assayed in animals using a standard protocol. In the assay, the candidate compound was prepared into a clear solution (intravenous injection) or a homogeneous suspension (oral administration) and administrated to the animal once. Whole blood samples were collected within 24 hours, and centrifuged at 3200 g for 10 minutes. The supernatant was separated to obtain a plasma sample. The blood concentration was quantitatively analyzed by LC-MS/MS analysis method, and pharmacokinetic parameters such as peak concentration, time to peak, clearance rate, half-life, and area under the drug-time curve were calculated.

TABLE 11
Pharmacokinetic parameters of the compound of the
present disclosure measured in various species

| | Compound No. | | | | Compound No. | | |
|---|---|---|---|---|---|---|---|
| | Compound 4 hydrochloride | | | | Compound 4 hydrochloride | | |
| | Mode of administration | | | | Mode of administration | | |
| | IV | | | | PO | | |
| Species | Mouse | Rat | dog | Species | Mouse | Rat | dog |
| Dosage (mpk) | 1 | 0.25 | 0.1 | Dosage (mpk) | 5 | 0.5 | 0.5 |
| CL | 0.98 | 4.1 | 7.2 | Cₘₐₓ (μM) | 31.0 | 0.53 | 0.27 |
| (mL/Kg/min) | | | | | | | |
| Vd (L/Kg) | 0.23 | 0.62 | 2.5 | Tₘₐₓ (μM) | 0.75 | 2.0 | 3.0 |
| T_{1/2} (h) | 2.6 | 2.2 | 6.2 | T_{1/2} (h) | 2.7 | 3.4 | 9.5 |
| AUC (μM · h) | 32.8 | 2.4 | 0.504 | AUC (μM · h) | 145 | 4.8 | 3.1 |
| AUC_{free} | 0.92 | 0.11 | 0.12 | AUC_{free} | 4.1 | 0.22 | 0.74 |
| (μM · h) | | | | (μM · h) | | | |
| | | | | F % | 88% | 103% | 121% |

Conclusion: The compound of the present disclosure has pharmacokinetic properties that meet the requirements for drug formulation, higher free drug exposure, and higher bioavailability in animals.

### Assay Example 6: Assay of Efficacy on Suckling Mice

Objective of assay: The in vivo antiviral activity of the compound against coronavirus was evaluated by observing the percentage of weight change and survival rate of the assay animals in a suckling mouse model infected with coronavirus (COV). The mouse infection model is widely used to determine the protective effect of the compound on virus-infected animals to reflect the antiviral activity of the compound.

### Assay Procedure:

Suckling mice (C57BL6/J strain) were inoculated with virus (coronavirus OC43) by intranasal drops on day 0, with an inoculation dose of 3500 p.f.u./mouse. From day 0 to day 6, the mice were treated with a vehicle (5% DMSO+40% polyethylene glycol 400+55% water) or 12.5 mpk or 25 mpk of the compound of the present disclosure for 7 consecutive days, once a day, by intraperitoneal injection, for a total of 7 times, with the first administration time being 2 hours before virus inoculation. The animals were continuously observed from day 0 to day 14, and the body weight, health and survival status were recorded.

The assay results are shown in Table 12:

TABLE 12
Protective effect of the compound of the present disclosure on
animals in the COV infection suckling mouse model (body weight)

| | Assay compound | | | | | |
|---|---|---|---|---|---|---|
| | Vehicle | | Compound 4 hydrochloride | | | |
| | Dose (mpk) | | | | | |
| | N/A | | 12.5 | | | |
| | Weight | | Weight | | 25 | |
| | changes | | changes | | Weight | |
| | (Percent of | | (Percent of | | changes | |
| | starting | | starting | | (Percent of | |
| | body | Survival | body | Survival | starting body | Survival |
| | weight) | rate | weight) | rate | weight) | rate |
| Day 1 | 8.06 ± 1.31 | 100% | 5.35 ± 0.58 | 100% | 6.45 ± 1.94 | 100% |
| Day 2 | 17.12 ± 1.24 | 100% | 14.57 ± 1.37 | 100% | 12.35 ± 1.74 | 100% |
| Day 3 | 25.14 ± 0.76 | 100% | 22.55 ± 1.37 | 100% | 22.61 ± 2.41 | 100% |
| Day 4 | 32.50 ± 0.68 | 100% | 30.95 ± 1.19 | 100% | 28.78 ± 2.69 | 100% |
| Day 5 | 36.35 ± 2.85 | 100% | 39.15 ± 1.15 | 100% | 34.72 ± 2.23 | 100% |
| Day 6 | 24.86 ± 4.72 | 83% | 42.61 ± 1.80 | 100% | 39.95 ± 2.01 | 100% |
| Day 7 | 7.14 ± 4.12 | 83% | 49.30 ± 2.16 | 100% | 45.35 ± 2.00 | 100% |
| Day 8 | 5.07 ± 5.52 | 33% | 55.56 ± 2.05 | 100% | 49.97 ± 1.91 | 100% |
| Day 9 | N/A | 0% | 57.80 ± 4.27 | 100% | 55.69 ± 2.31 | 100% |
| Day 10 | N/A | 0% | 60.74 ± 6.34 | 100% | 62.00 ± 2.26 | 100% |
| Day 11 | N/A | 0% | 60.39 ± 8.87 | 100% | 72.13 ± 3.02 | 100% |
| Day 12 | N/A | 0% | 65.10 ± 10.85 | 100% | 87.49 ± 3.63 | 100% |
| Day 13 | N/A | 0% | 76.18 ± 9.69 | 100% | 95.42 ± 4.90 | 100% |
| Day 14 | N/A | 0% | 96.72 ± 10.96 | 100% | 118.37 ± 5.15 | 100% |

N/A: Not applicable here

Conclusion: The compound of the present disclosure shows protective effects in the efficacy assay in the animal model.

1. A compound represented by formula (VI) or a pharmaceutically acceptable salt thereof, 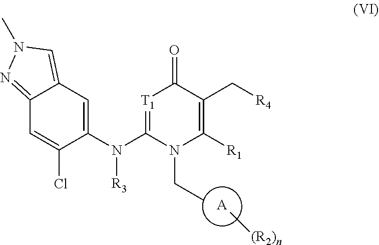
wherein
R₁ is selected from H, F, Cl, Br, I and C₁₋₃ alkyl, wherein the C₁₋₃ alkyl is optionally substituted with 1, 2 or 3 halogens;
each R₂ is independently selected from H, F, Cl, Br, I and C₁₋₃ alkyl, wherein the C₁₋₃ alkyl is optionally substituted with 1, 2 or 3 halogens;
Ring A is selected from phenyl and 5- to 6-membered heteroaryl;
R₃ is selected from H and C₁₋₄ alkyl;
R₄ is selected from 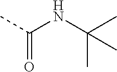
and 5-membered heteroaryl, wherein the 5-membered heteroaryl is optionally substituted with 1 or 2 Rₐ;
each Rₐ is independently selected from F, Cl, Br, I, C₁₋₃ alkyl and C₃₋₆ cycloalkyl, wherein the C₁₋₃ alkyl and C₃₋₆ cycloalkyl are optionally substituted with 1, 2, or 3 halogens;
n is selected from 1, 2, 3, 4 and 5;
T₁ is selected from CH and N.

2. The compound according to claim 1 or a pharmaceutically acceptable salt thereof, wherein R₁ is selected from H, F, —CH₃ and —CF₃.

3. The compound according to claim 1 or a pharmaceutically acceptable salt thereof, wherein each R₂ is independently selected from H, F, —CH₃ and —CF₃.

4. The compound according to claim 1 or a pharmaceutically acceptable salt thereof, wherein R₃ is selected from H and —CH₃.

5. The compound according to claim 1 or a pharmaceutically acceptable salt thereof, wherein R₄ is selected from 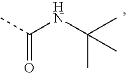
pyrrolyl, imidazolyl, pyrazolyl and triazolyl, wherein the pyrrolyl, imidazolyl, pyrazolyl and triazolyl are optionally substituted with 1 or 2 Rₐ.

6. The compound according to claim 5 or a pharmaceutically acceptable salt thereof, wherein R₄ is selected from

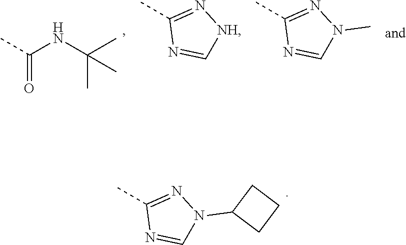

7. The compound according to claim 1 or a pharmaceutically acceptable salt thereof, wherein ring A is selected from phenyl, pyridyl, pyrimidinyl, pyridazinyl, pyrazolyl, pyrrolyl and imidazolyl.

8. The compound according to claim 1 or a pharmaceutically acceptable salt thereof, wherein ring A is selected from phenyl,

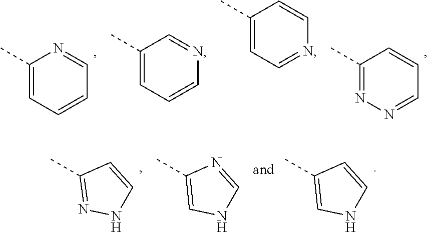

9. The compound according to claim 1 or a pharmaceutically acceptable salt thereof, wherein the structural moiety 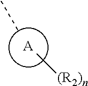
is selected from

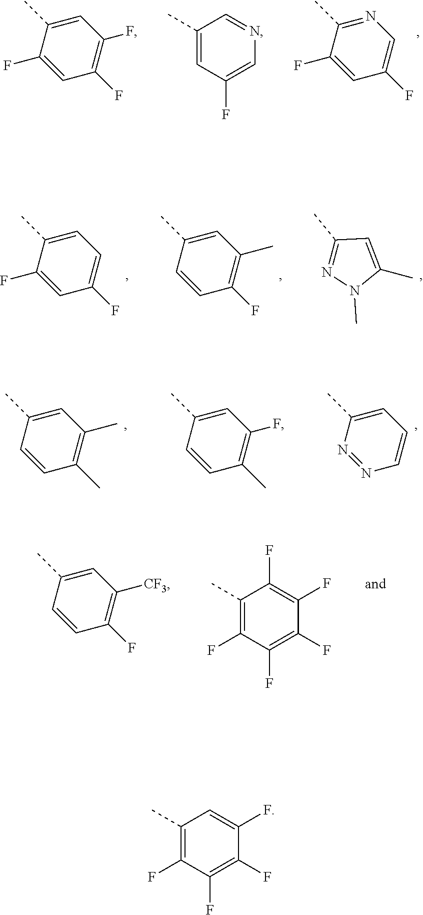

10. The compound according to claim 1 or a pharmaceutically acceptable salt thereof, wherein the compound has the structure represented by formula (VI-1): 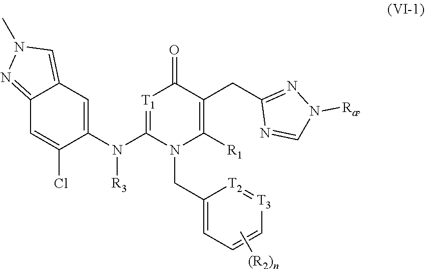
wherein T₂ and T₃ are each independently selected from CH and N;
T₁, R₁, R₂, R₃, Rₐ and n are as defined in claim 1.

11. A compound of the following formulae or a pharmaceutically acceptable salt thereof,

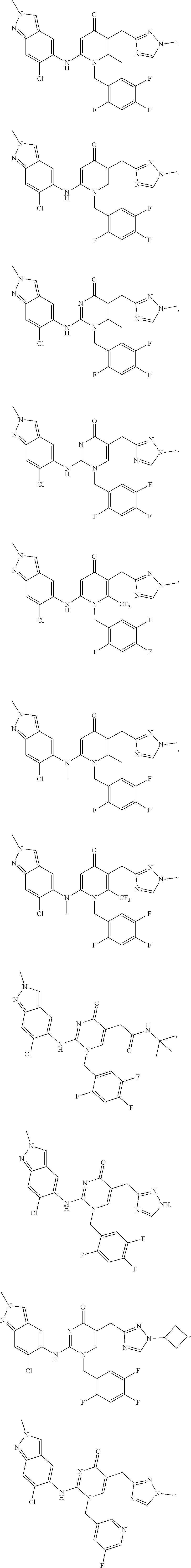

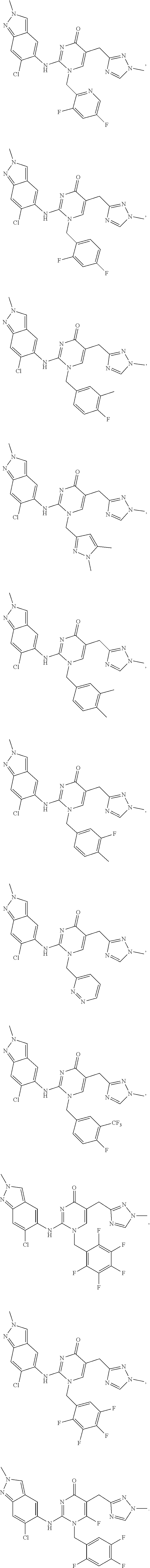

12. A method of treating novel coronavirus infection in a subject in need thereof, comprising administering to the subject the compound according to claim 1 or a pharmaceutically acceptable salt thereof.

13. A method of treating novel coronavirus infection in a subject in need thereof, comprising administering to the subject the compound according to claim 11 or a pharmaceutically acceptable salt thereof.
